(19) United States
(12) Patent Application Publication
(10) Pub. No.: US 2026/0325040 A1
(43) Pub. Date: Oct. 22, 2026

(54) SELECTIVELY RECORDING HIGH-FIDELITY DATA BY A MEDICAL DEVICE

(71) Applicant: Stryker Corporation, Portage, MI (US)

(72) Inventors: Adam Harvey, Hillsborough (GB); Fred W. Chapman, Newcastle, WA (US)

(73) Assignee: Stryker Corporation, Portage, MI (US)

(21) Appl. No.: 19/654,321

(22) Filed: Apr. 21, 2026

Related U.S. Application Data
(60) Provisional application No. 63/792,496, filed on Apr. 22, 2025.

(51) Int. Cl. A61B 5/333; A61B 5/00; A61B 5/339; A61N 1/02; A61N 1/39; G16H 40/67

(52) U.S. Cl. CPC A61B 5/333; A61B 5/0006; A61B 5/339; A61N 1/025; A61N 1/3904; A61N 1/3925; A61N 1/3987; A61N 1/3993; G16H 40/67

(57) ABSTRACT

An example method includes continuously recording high-fidelity data at a medical device in a circular buffer. The high-fidelity data can include physiological parameter data, user operational data, and medical device data. The method also includes detecting a trigger event. In response to detecting the trigger event, the medical device can store, in a memory associated with the medical device, a portion of the high-fidelity data corresponding to a period of time after the trigger event. The method further includes transmitting the portion of the high-fidelity data to an external device.

### CROSS-REFERENCE TO RELATED APPLICATION

This application claims priority to U.S. Provisional App. No. 63/792,496, which was filed on Apr. 22, 2025 and is incorporated by reference herein in its entirety.

### BACKGROUND

Medical devices can be used to monitor patients and/or to administer treatments to patients. Medical devices used in emergency situations must enable rapid and correct use for timely treatment of patients experiencing life-threatening emergencies. However, issues can arise during use and it is not always clear if or how a medical device is being misused. Current sources of information that may provide insight into the use of a medical device are limited.

### BRIEF DESCRIPTION OF THE DRAWINGS

FIG. 1 illustrates an example environment for recording data generated by a medical device(s) and transmitting a recorded file including the data to an external computing device.

FIG. 2 illustrates example signaling for facilitating communication between devices described with reference to FIG. 1.

FIG. 3 illustrates an example of a recorded file described with reference to FIGS. 1 and 2.

FIG. 4 illustrates an example process for recording data, detecting a trigger event, storing a portion of the recorded data, and transmitting the portion of the recorded data to an external device.

FIG. 5 illustrates an example process for determining a type of medical device that is recording data and determining to record the data at or above a threshold sample rate.

FIG. 6 illustrates an example process for detecting a trigger event indicating a first setting associated with a first medical device that is communicatively coupled to a second medical device is incompatible with a second setting associated with the second medical device.

FIG. 7 illustrates an example of an external defibrillator configured to perform various functions described herein.

### DETAILED DESCRIPTION

To improve medical devices and/or to coach users on proper use, it is important to understand the circumstances of how issues arise during their use. Gathering data indicating how a medical device is used can be resource intensive. Previous sources of information regarding use of medical devices are limited to user interviews, observation of simulated events, and/or examination of medical device data associated with complaints.

When issues do occur, it is challenging if not impossible to understand what the medical device (or user) did or why. It is also difficult to test all possible scenarios during the design and manufacture of a medical device. Additionally, many scenarios are not testable (e.g., in the case where a user misses an alert or error presented via the display). Given this, it can be difficult to understand a root cause of an issue. Medical devices can store a variety of data during use. However, storage on a medical device generally has limited capacity and transmission of very large data files is time consuming and expensive. As such, storing all possible data generated and/or received at the medical device is inefficient, particularly if the data is high-fidelity data. As used herein, “high-fidelity data” can include data that is recorded at or above a threshold sample rate (or frequency), data that is recorded at or above a threshold resolution, data that is recorded at or above a threshold accuracy (e.g., data that has level of noise at or below a threshold), precision, and/or level of detail.

Various implementations described herein relate to recording (or otherwise capturing) and storing data in memory of a medical device during operation. The data can be recorded in a file or other data structure. In some examples, the data can be recorded at a higher fidelity by, for example, increasing a sample rate of recorded signals and/or changing a filtering bandwidth of signals being recorded to allow capture of higher and lower frequency components of the signals. In some cases, the recorded file can capture parameters and signals not usually stored by the medical device, such as scene audio captured during a rescue event, data that was displayed on the screen for a period of time (e.g., a video recording of the user interface), etc. The medical device can be configured to continuously store a specified quantity of data (e.g., a maximum or threshold amount of data) temporarily in a circular buffer. That is, the oldest data can be overwritten as additional data is stored in memory. The medical device may be configured to store (e.g., in long-term and/or non-volatile storage) and/or transmit the data upon detecting a trigger event. Trigger events that initiate a detailed, black-box recording may include events that are useful for manufacturers of the medical device and/or users of the medical device. The data can be analyzed in order to better understand the circumstances of the event. In this manner, a portion of data that is related to an event of interest (e.g., an anomalous event) is stored and/or transmitted to an external device for review and/or processing.

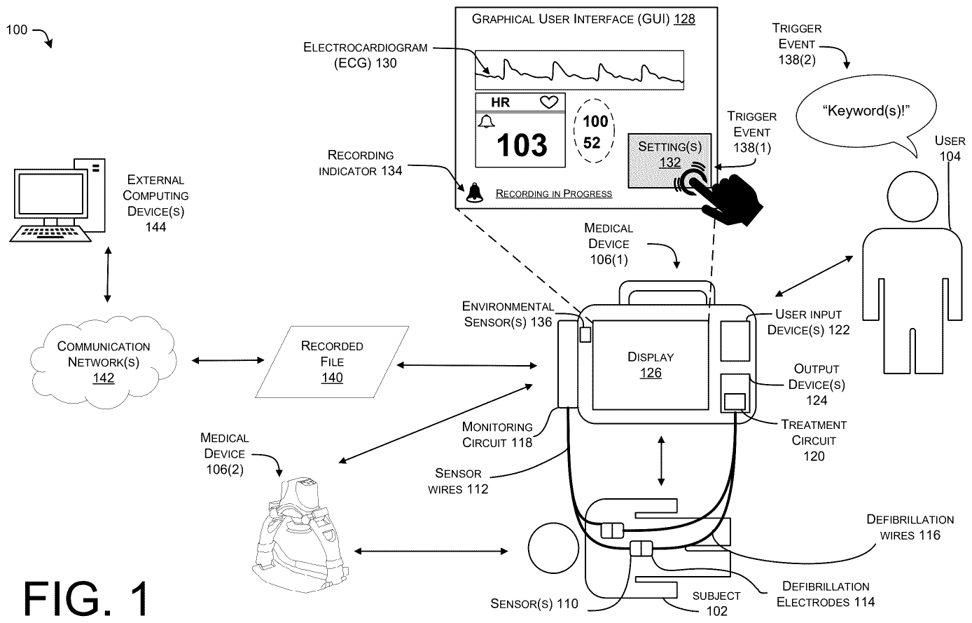

FIG. 1 illustrates an example environment 100 for recording data generated by a medical device(s) (at the device level) and transmitting a recorded file including the data to an external computing device(s) 144. As illustrated, the environment 100 includes a rescue scene. In some examples, the rescue scene may be in a clinical environment (e.g., a hospital) or a non-clinical environment (e.g., the scene of an accident). In some cases, the subject 102 may be experiencing cardiac arrest, respiratory arrest, or some other dangerous medical condition. The subject 102 is being monitored and/or treated by a user 104 (e.g., an emergency medical technician (EMT) professional, care provider, etc.) utilizing one or more medical devices. The user 104 may be utilizing one or more medical devices to evaluate, treat and/or monitor a condition of the subject 102. For example, the user 104 may utilize a first medical device 106(1) (e.g., a monitor-defibrillator) and a second medical device 106(2) (e.g., a chest compression device). In some instances, additional users may be assisting user 106. Although illustrated in FIG. 1 as separate individuals, in some cases, the subject 102 is the user 106.

According to some implementations, the medical device 106(1) is a defibrillator. For example, the medical device 106(1) may be an external defibrillator, such as an automated external defibrillator (AED) or a monitor-defibrillator. In some examples, the medical device 106(1) may be a portable medical device. A user 104 may utilize the medical device 106(1) to evaluate, treat, and/or monitor a condition of the subject 102. In some examples, the user 104 may utilize additional medical device(s) (e.g., medical device 106(2)) to evaluate, treat, and/or monitor the subject 102. For example, the user 104 may utilize one or more of an additional monitor-defibrillator, a non-invasive blood pressure (NIBP) monitor, a medical imaging device, an ultrasound monitor, a standalone ECG monitor, chest compression device, ventilator, pulse oximeter, capnograph, continuous glucose monitor (CGM), blood pressure monitor, portable X-ray machine, transport cot or bed, medical cot, implantable device such as a cardioverter-defibrillator (ICD), pacemaker, plethysmograph, and/or any other medical device or equipment configured to assess, treat, and/or monitor an individual.

The medical device 106(1) (e.g., a monitor-defibrillator) may include sensor(s) 110 (e.g., parameter sensor(s), sensor electrodes, environmental sensor(s) 136, etc.), a monitoring circuit 118, a treatment circuit 120, an output device(s) 122, a user input device(s) 124 and/or a display 126 presenting a graphical user interface (GUI) 128.

A monitoring circuit 118 can detect one or more physiological parameters of the subject 102 via the sensor(s) 110. As used herein, the term “physiological parameter,” and its equivalents, may refer to an indication of a subject's health including, for instance, an electrocardiogram (ECG) 130, an impedance (e.g., transthoracic impedance), a force administered to the subject 102, a blood pressure, an airway parameter (e.g., a partial pressure of carbon dioxide, a partial pressure of oxygen, a capnograph, an end tidal gas parameter (e.g., end-tidal CO₂ (EtCO₂)), a flow rate, etc.), a blood oxygenation (e.g., a pulse oximetry value, a regional oximetry value (e.g., cerebral regional tissue oxygen saturation), etc.), an electroencephalogram (EEG), an electromyogram (EMG), a temperature, a heart sound, a blood flow rate, a physiological geometry (e.g., a shape of a blood vessel, an inner ear shape, etc.), a heart rate, a pulse rate, a chest compression position, changes in volume within an organ, or the like.

The monitoring circuit 118 includes and/or is communicatively coupled to a sensor(s) 110. The sensor(s) 110 may be configured to detect at least one physiological parameter of the subject 102. For example, a sensor(s) 110 may include at least one of electrodes, a detection circuit, defibrillation electrodes 114, a force sensor, a blood pressure cuff, an ultrasound-based blood pressure sensor, an invasive (e.g., intra-arterial) blood pressure sensor (e.g., including a cannula inserted into the subject 102), a gas sensor (e.g., a carbon dioxide and/or oxygen sensor), a flowmeter, a pulse oximetry sensor, a regional oximetry sensor, etc. In some instances, the medical device 106(1) may include a thermometer and/or a microphone.

In some cases, the physiological parameters indicate a condition of a heart of the subject 102. According to various examples, the physiological parameters include a voltage generated by the heart of the subject 102, a pulse oximetry level of the subject 102, or any combination thereof. According to some examples, the physiological parameters detected by the monitoring circuit 118 include an electrical signal output by the heart of the subject 102. For example, sensor(s) 110 receive an electrical signal (e.g., the voltage) output by the heart of the subject 102. The sensor(s) 110 may be adhered to the subject 102 via an adhesive. The sensor(s) 110 receive an electrical signal (e.g., the voltage) output by the heart of the subject 102. Sensor wires 112 electrically couple the sensor(s) 110 to the monitoring circuit 118. In some cases, the monitoring circuit 118 is configured to convert the electrical signal into digital data indicative of an ECG 130 (also referred to as an “ECG signal”) of the subject 102. Although FIG. 1 illustrates a pair of sensor(s) 110, in some examples, the monitoring circuit 118 receives electrical signals indicative of the ECG 130 from three or more sensor electrodes. For example, a 12-lead ECG is obtained from ten sensor electrodes placed on the skin of the subject 102. In some examples, the monitoring circuit 118 includes an analog to digital converter.

In some examples, the monitoring circuit 118 includes one or more ports configured to receive analog signals indicative of the physiological parameters. The one or more ports can be electrically coupled to one or more sensor(s) 110 (e.g., parameter sensor).

The medical device 106(1) may include a display 126 configured to graphically output physiological parameters, alerts, messages, instructions, etc. As shown in the example of FIG. 1, the display 126 outputs a GUI 128 that includes at least a portion of the ECG 130 of the subject 102. The ECG 130 includes a first axis corresponding to time and a second axis corresponding to voltage. In some cases, the GUI 128 includes multiple plots corresponding to different (e.g., 12) leads of the ECG 130. In some instances, the display 126 may present an alert, instruction, recommendation, etc. to the user 104. In some examples, the alert may be a visual and/or aural alert. In some instances, the GUI 128 may notify users of a detected artifact, types of artifacts, lead(s) affected, and electrodes causing a problem, lead wire reversal, if present, or any other type of problem, warning, recommendation, instruction, etc. The display 126 may present physiological parameters in a manner that is easily digested and understood while also conveying a reliability of such data.

The medical device 106(1) includes a user input device(s) 122 and an output device(s) 124. The user input device(s) 122 and the output device(s) 124 function as an interface between a user and the medical device 106(1). The input device(s) 122 is configured to receive an input from a user and includes at least one of a keypad, a cursor control, a touch-sensitive display, a voice input device (e.g., a microphone), a haptic feedback device (e.g., a gyroscope), or any combination thereof. The output device(s) 124 includes at least one of a display 126, a speaker, a haptic output device, a printer, or any combination thereof. In some implementations, the input device(s) 122 includes one or more touch sensors, and the output device(s) 124 includes a display screen, and the touch sensor(s) are integrated with the display screen.

In at least one example, the medical device 106(1) is a monitor-defibrillator configured to output a defibrillation shock and/or a pacing signal to the subject 102. The output device(s) 124 is connected to a set of defibrillation wires 116. The defibrillation wires 116 are connected to defibrillation electrodes 114 that are in contact with the skin of the subject 102. According to some examples, the defibrillation electrodes 114 are integrated with, or at least partially in contact with, at least some of the sensor(s) 110. In some cases, the output device(s) 122 includes a treatment circuit 120 configured to selectively output a voltage (e.g., a defibrillation shock) across the defibrillation electrodes 114. For example, the output device 124(s) may include a capacitor configured to store a voltage and a discharge circuit configured to discharge the voltage across the defibrillation electrodes 114. The voltage is applied over the heart of the subject 102 and depolarizes cells within the heart. The voltage may cause the heart to, at least eventually, restore a healthy heart rhythm.

In some examples, a second medical device 106(2), such as a mechanical chest compression device as illustrated in FIG. 1, may be utilized in conjunction with the first medical device 106(1) in order to assist with the monitoring and/or treatment of the subject. For example, the second medical device 106(2) may include any other medical device configured to detect a parameter of the subject 102, or administer a treatment (e.g., an electrical shock, pacing, chest compressions, etc.) to the subject.

In some examples, the medical device 106(1) may include an environmental sensor(s) 136 configured to detect an environmental parameter indicative of an environment surrounding the medical device 104(1), such as during a rescue event, during transport (e.g., movement of the medical device 104(1) to and from the rescue scene), during storage, or any combination thereof. The environmental sensor(s) 136 may include a temperature sensor configured to measure a temperature of the environment external to the medical device 106(1). In some instances, environmental temperature may provide insight into how the medical device 104(1) functions and/or is used. In various examples, extreme temperatures (both hot and cold) may play a role in the performance of the medical device 106(1) and/or accessory devices stored with the medical device 106(1). For example, relatively high temperatures (e.g., over 35 degrees Celsius, such as in Arizona) may degrade internal circuit components of the medical device 106(1), degrade an electrically conductive gel of the sensor(s) 110 during storage, or cause other damage that may interfere with the function of the medical device 106(1). In some cases, relatively cold temperatures (e.g., under 0 degrees Celsius, such as in Alaska) may cause circuit components of the medical device 106(1) to break or may reduce their conductivity, which may also cause the medical device 106(1) to malfunction during use.

In some cases, the environmental sensor(s) 136 may include a humidity sensor (e.g., a hygrometer) configured to measure a humidity of the environment external to the medical device 106(1). In some instances, a humidity level of the environment may affect the performance of the medical device 106(1) and/or a condition of the subject 102 during the rescue event. Humidity, for instance, may promote corrosion of electronic components and/or circuitry in the medical device 106(1), which can lead to electrical shorts, intermittent connections, and/or permanent damage of components of the medical device 106(1). In some cases, humidity can impact the function of accessory devices. For example, humidity can impact the calibration and/or accuracy of a light-based sensor for detecting an amount of CO₂ in the airway of the subject 102. Thus, a medical device 106(1) operating in a relatively humid environment (e.g., Hawaii) may operate differently than if the medical device 106(1) is operating in a relatively dry environment (e.g., Arizona).

In various examples, environmental characteristics, such as temperature and/or humidity, may affect how a sensor(s) 110 functions. In some cases, environmental characteristics of the medical device 106(1) impact how effectively an adhesive adheres the sensor(s) 110 and/or the defibrillation electrodes 114 to the subject 102. In some examples, environmental characteristics impact how effectively a suction cup of a chest compression device adheres to the subject 102. In some cases, environmental temperature may affect the performance and/or lifespan of batteries used in the medical device 106(1) (e.g., high temperatures can accelerate battery degradation and reduce capacity while low temperatures can decrease battery efficiency and cause voltage fluctuations). In some instances, sensor accuracy may be affected by changes in ambient temperatures (e.g., variations in accuracy or calibration due to changes in ambient temperatures, which affect reliability and precision of measurements of data collected by the medical device 106(1)). Rapid changes in temperature and/or humidity may cause ingress of moisture which can lead to material degradation of plastics, adhesives, seals, etc. which can lead to warping or deterioration of the medical device 106(1). In some instances, environmental data may indicate or be associated with a misuse of the medical device 106(1) (e.g., improper storage).

In some cases, the environmental sensor(s) 136 may include an accelerometer configured to detect movement (e.g., vibration, orientation, etc.) of the medical device 106(1) and/or an accessory. An accessory can include, for example, an ECG/EKG electrode, lead wires, defibrillator pad, blood pressure cuff, pulse oximeter probe, syringe pump, and the like. The accelerometer data can be used to determine whether the medial device 106(1) has been damaged (e.g., hit), dropped, or thrown, whether the medical device 106(1) was being moved during treatment (e.g., during administration of a shock), and the like. Analysis of data indicative of the environmental condition(s) may indicate that the medical device 104(1) was exposed to extreme conditions (e.g., improper storage) which can lead to unsafe operation. In some cases, the analysis of the data can identify types of environmental conditions that cause unsafe or unreliable operation of the medical device 104(1), or other similar medical devices. As such, gathering and analyzing environmental data may provide insight into how medical devices may be improved.

In some examples, the medical device 106(1) can include a microphone configured to capture ambient audio data. The ambient audio data can be recorded from the environment surrounding the medial device 106(1). Ambient audio data may indicate environmental sounds surrounding the medical device 106(1) (e.g., traffic noise, music, etc.), human activity (e.g., coughing, breathing patterns (e.g., irregular breathing pattern, increase breathing rate, etc.), spoken keywords, etc.), acoustic levels (e.g., a noise level in the surrounding environment), and the like. In some instances, the medical device 106(1) may be configured to cause a microphone to capture ambient audio data during a rescue event in response to detecting a trigger event. The ambient audio data may be stored in a memory of the medical device 106(1) (e.g., in a recorded file 140) and/or transmitted to an external computing device 144.

The first medical device 106(1) and/or the second medical device 106(2) may be configured to identify, generate, and/or receive different types of data. For example, data can include parameter data, operational data, user input data, etc. As used herein, receiving data may refer to the process of obtaining or acquiring data from an external source such as sensors, instruments, networks, or other devices. The received data may be raw data or preprocessed data and may be in various forms (e.g., digital signals, analog signals, text, images, audio, video, etc.). As used herein, identifying data may refer to recognizing or determining the characteristics, attributes, and/or patterns of the data received. Identification of data may include parsing, classifying, labeling, and/or categorizing the data based on a predefined criteria, parameter, or rule. The process of identifying data may assist in distinguishing relevant data from noise, extract meaningful features, and organize the data for analysis. In particular examples, the medical device 106(1) may be configured to annotate the data at the device level prior to transmitting the annotated data to an external computing device(s). In some instances, annotating the data may include labeling or adding metadata to the data to make it usable by machine learning algorithms. As used herein, generating data may refer to the process of creating or producing new data based on existing information or through computational processes. Generating data can involve modeling, synthesis, annotating, and/or transformation of existing data into new forms or representations. For example, generating new data may include generating encrypted data by encrypting privileged data using an encryption algorithm and a unique encryption key. In some instances, the data may be grouped and/or clustered using various techniques and algorithms (e.g., using K-means clustering techniques, hierarchical clustering techniques, density-based spatial clustering techniques, gaussian mixture models, spectral clustering, agglomerative clustering, self-organizing maps (SOM) techniques, and the like).

The medical device 106(1) may be configured to continuously record data in a circular buffer. For example, the data may be recorded in a data structure (e.g., linear queue format) that follows a first-in, first-out principle. That is, the oldest data is discarded or overwritten by the newest data. The data may be recorded in a pre-determined (or fixed-size) block of memory (e.g., a predetermined memory buffer). In some instances, the block of memory may have between about 4 GB to about 1 TB in storage capacity. In some examples, the storage capacity of the pre-determined block of memory may be based on the type of medical device and/or storage capacity associated with the device (e.g., a monitor-defibrillator may have a larger pre-determined block of memory relative to an NIBP device).

In some examples, the GUI 128 of the display 126 may be configured to present a user interface element (e.g., recording indicator 134) indicating the medical device 106(1) is actively recording data. In some examples, the medical device 106(1) may cause the display to present the recording indicator 134 in response to detecting a trigger event.

The medical device 106(1) may be configured to continuously record high-fidelity data and/or low-fidelity data. High-fidelity data can include data that is recorded at or above a threshold sample rate (or frequency), data that is recorded at or above a threshold resolution, data that is recorded at or above a threshold accuracy (e.g., data that has level of noise at or below a threshold), precision, and/or level of detail. Low-fidelity data can include data that is recorded at or below a threshold sample rate (or frequency), data that is recorded at or below a threshold resolution, data that is at or below a threshold accuracy, etc. In at least one example, low-fidelity data may be data that is routinely generated and/or transmitted by the medical device. For example, physiological signals recorded at a standard sampling frequency useful for interpretation of an underlying physiology (e.g., diagnostic ECG recorded at a sampling frequency between about 125 Hz to about 500 Hz). In some instances, recording data at a lower sampling frequency may be insufficient for troubleshooting a complex issue. For example, data that is recorded below a threshold sample rate may not record narrow pacer spikes (or abnormal electrical signals) that appear on an ECG when an implanted medical device (e.g., pacemaker) regulates a heartbeat. To provide a non-limiting example, high-fidelity data may include signals recorded from all available ECG leads (e.g., 12 ECG leads) connected to the subject 102 while low-fidelity data may include signals received from less than all available leads (e.g., less than 12 ECG leads).

The medical device 106(1) may be configured to record various types of data. The recorded data may be associated with timestamps. The data can include physiological parameter data (e.g., heart rate, blood pressure, oxygen saturation, etc.), user operational data (e.g., button presses, touch screen taps/swipes, keyboard input, etc.), and/or device data (e.g., alerts, alarms, instructions generated by the device, display data, etc.). Data may be recorded in one or more data channels. Data channels can include parameter data channels including physiological signals recorded at one or more sample rates (or within a range of sample rates). For example, an ECG data channel including signals recorded between about 125 Hz and about 500 Hz, SpO₂ data channel including signals recorded between about 1 Hz (e.g., every second) and about 200 Hz. In some instances, oxygen levels may be recorded at about 1 Hz while the pulse oximeter waveform data can be recorded at a sample rate of between about 30 Hz to about 200 Hz. A blood pressure channel can include signals recorded at a predetermined time interval (e.g., every 1 second, 30 seconds, 1 minute, etc.) or a sample rate of between about 100 Hz to about 200 Hz, respiratory data channel including signals recorded at a sample rate between about 5 Hz and 250 Hz, temperature data channel including signals recorded at a predetermined time interval (e.g., every 1 second, 30 seconds, 1 minute, etc.), EEG data channel including signals recorded between about 500 Hz and about 2000 Hz, and the like. Data channel(s) can include waveform data channel(s) indicating raw or processed signal data that is presented via a GUI (e.g., ECG waveforms, EEG traces, capnography waveforms, etc.).

In some instance, data channel(s) can include an event data channel including data indicating an alarm was triggered (e.g., detecting a heart rate is above at or above a threshold heart rate), a manual event (e.g., user pressed a button), system event (e.g., battery level low). Data channel(s) may include a configuration channel(s) indicating various configurations of the medical device, such as patient demographics, sensor type(s) and placement, etc. Data channel(s) may include control/command channels including signals indicating mode or setting switches (e.g., switch from adult mode to pediatric mode, start/stop commands, etc.).

The medical device 106(1) may record device data. Device data can include output signals generated by the medical device. Device data can include, for example, data generated and/or received by the medical device, such as physiological data captured from the subject 102, device status data indicating a state of the medical device, and the like. Device data can indicate a battery level, an operational mode the device is in (e.g., standby or active), firmware/software version of the device, component status (e.g., sensor connected/disconnected), error codes or fault logs, diagnostic data, configuration and/or setting data (e.g., alarm thresholds, measurement intervals, etc.), self-test log(s) and/or calibration results, communication and connectivity data (e.g., network status, BLUETOOTH™/WI-FI™, pairing logs, etc.), and the like.

In some examples, device data can include display data indicating information that is visually presented on the display during a trigger event. In some instances, the medical device 106(1) may record a portion of the data presented via the display.

The medical device 106(1) may record user operational data (or user input data) indicating how a user interacted with the medical device. User operational data may include user input signals detected by the medical device. For example, user input data can include data indicating button presses (mechanical or capacitive), knob or dial rotations, switch toggles, slider input, touch screen taps or swipes, keyboard input, mouse input (or pointer-based interactions), physical attachments such as inserting a probe or attaching a sensor, pugging in a cable or other connector, and the like. In some instances, user input data can include voice commands or keywords detected by a microphone (e.g., a microphone disposed on the medical device or via a microphone that is communicatively coupled to the medical device). To provide a non-limiting example, the medical device 106(1) can record data indicating button presses associated with switching between an adult mode and a pediatric mode. In some instances, the recorded data may indicate that a user repeatedly presses pediatric mode (or held a button for a threshold period of time), but the medical device does not switch modes.

In some examples, a user may be wearing a device, such as a smartwatch, body-worn camera, and the like. The wearable device can record data, such as motion data (e.g., detect that a user is walking), physiological data (e.g., heart rate, etc.), sound data (e.g., via a microphone on the wearable device), etc. In some examples, the medical device 106(1) may be communicatively coupled to the wearable device associated with the user 104. The medical device 106(1) may receive data recorded at the wearable device and store a portion of the data received from the wearable device. In some instances, data received from the wearable device may be stored in association with a trigger event.

The medical device 106(1) may record data indicating use of the medical device, such as data input by a user (e.g., patient data input by a user), a user response to an alert generated by the medical device (e.g., a user may silence an alarm output by the medical device), a time at which the user response was detected, a user response to a prompt generated by the medical device (e.g., the medical device may prompt the user to initiate treatment), an indication of a selection of one or more user interface elements, buttons, etc. of the medical device in response to a notification output by the medical device, and the like. In some instances, use of the medical device may include data indicating whether the medical device was dropped, whether the medical device was being moved during treatment (e.g., during administration of a shock), whether the medical device was shaking during treatment or use, and the like. For example, the medical device may include one or more of an accelerometer sensor, gyroscope sensor, magnetometer sensor, linear acceleration sensor, rotation vector sensor, etc. that enable the medical device to gather data related to whether the medical device is shaking or being moved during use. To provide one non-limiting example, a medical device may include an accelerometer that measures the acceleration forces acting on the medical device in three dimensions (e.g., x, y, and z axes) and can detect movement of the device (e.g., whether the medical device was being shaken, tilted, or was dropped).

The medical device 106(1) may record eye movement data indicating where, when, and/or how a user's eyes move. The medical device 106(1) may include a camera sensor positioned proximate the display. The camera sensor can capture images of the eyes in order to track movement and gaze direction. For instance, the medical device 106(1) can include a near-infrared light illumination module including a light emitter that emits near-infrared spectrum light. The images are processed to locate a center of a pupil and the light reflection of the cornea of the eye. In some examples, an image processing algorithm and/or an eye tracking algorithm can be used to identify the center of the pupil and infer the location of a point of gaze.

In some instances, the medical device 106(1) may be configured to record data associated with the subject 102 and/or the user 104. For example, the medical device 106(1) may record an identifier of the subject 102 including information used to identify the subject or patient. An identifier of a subject may include a subject name, subject identification number (e.g., patient identification number, medical record number, health insurance number, social security number, etc.), sex, age, weight, medical history, physiological data, date of birth, address, phone number, biometric information (e.g., fingerprints, iris scans, facial recognition, etc.), and the like. The medical device 106(1) may receive and/or generate data including an identifier of the user (e.g., the rescuer's name, electronic identification number, biometric data, medical device purchaser information (e.g., a hospital, organization, etc.) and the like. The medical device 106(1) may be configured to generate, identify, and/or receive data representing a location of the medical device 106(1) (e.g., geographic coordinates, address, elevation, etc.) using one or more sensor(s) and/or systems (e.g., global positioning system (GPS) sensor, global navigation satellite system, another type of navigation satellite system, WI-FI™ positioning system (WPS), cellular triangulation or WLAN, etc.) which may be indicative of the identity of the subject 102. The medical device 106(1) may generate, receive, and/or identify data representing a time of a rescue event.

The medical device 106(1) may be configured to detect a trigger event (e.g., trigger event 138(1) and trigger event 138(2)). Detecting a trigger event(s) can cause the medical device to store a detailed, black-box recording of event(s) that may be useful to a manufacturer of the medical device and/or users of the medical device. That is, the trigger event may be an event of interest, such as an event that indicates the device is malfunctioning, being misused, etc. This data may be utilized to improve a functioning of the medical device(s) (e.g., improve algorithms, alerts, etc.), educate users of the medical device, and/or improve training of personnel that use the medical device, and the like.

In some examples, the trigger event may include a determination that a particular feature, setting, and/or mode of the medical device 106(1) is active or is otherwise being used (or combination of features, settings, and/or modes). For example, the trigger event may be a determination that the medical device 106(1) is generating electrical impulses to stimulate the heart of the subject 102 to contract (e.g., pacing), delivering a shock synchronized with the R-wave (e.g., QRS complex) on the ECG to treat tachyarrhythmia like atrial fibrillation (e.g., synchronized cardioversion), and the like.

In some instances, the trigger event may include a determination that a feature, setting, or mode of the medical device 106(1) is being used unsuccessfully. For example, a pacemaker can initiate a stimulus (or pacing spike), but the heart muscle may not be responding to it. The pacing spike may be detected in the ECG, but no subsequent QRS complex (ventricular depolarization), P wave (atrial depolarization), or blood pressure pulse follows. The medical device 106(1) may be set to a “synchronize” mode by pressing an appropriate button on the medical device 106(1). To provide another example, the medical device 106(1) can determine that unintentional induction of ventricular fibrillation (VF) during synchronized cardioversion occurred if a shock is delivered during the vulnerable period of ventricular repolarization (T-wave), which may occur due to inaccurate synchronization or other factors. The medical device 106(1) may detect that shocks are delivered using a synchronized cardioversion mode, followed by subsequent shock attempts delivered while in a mode not intended for cardioversion.

In some examples, the trigger event may include detection that the medical device 106(1) transitioned between modes or settings in a manner that is unexpected or atypical. For example, the medical device 106(1) may determine that the medical device 106(1) was in a first mode (e.g., adult mode) for a period of time (e.g., 10 minutes) and was subsequently changed to a different mode (e.g., pediatric mode). In some instances, the second mode or setting may be incompatible with the first mode. For example, when using an AED, the AED may default to an adult mode having settings that enable the AED to deliver high energy shocks. However, when using an AED on children (e.g., 8 years old or younger), it is generally recommended to perform defibrillation with attenuated settings by activating a pediatric mode on the AED.

In some examples, the trigger event may include detection of an artifact. For instance, an artifact in data representative of a physiological parameter may indicate that a physiological signal associated with a physiological parameter was detected by an incorrect sensor device (e.g., a sensor device designed for an adult used on a patient who is a child, a sensor device that is improperly connected to the medical device, etc.). In some examples, an artifact may be a physiological parameter artifact such as a cardiac artifact (e.g., a EEG artifact), an artifact caused by the presence of metal objects or variations in magnetic fields (e.g., electromagnetic interference), technical artifacts caused by a malfunction of the medical device (e.g. sensor saturation or clipping due to signal exceeding a measurable range, analog-to-digital converter quantization noise, loose or dried-out electrodes, etc.) artifacts caused by muscle activity or motion (e.g., caused by the subject 102 moving or being moved during operation of the device), a sensor being displaced, loose leads, vibration of the device, and the like. In some instances, the trigger event may include identifying an artifact appearing in a signal recorded by the medical device 106(1). For example, the medical device 106(1) may detect an artifact previously indicated to be associated with modified sine wave inverters, which can cause artifacts related to buzzing of humming of audio, harmonic distortion and the like. To provide another non-limiting example, the medical device may be a pacemaker and the trigger event is detection of a threshold number of signals (e.g., impulses) indicating an artifact (e.g., due to electromagnetic interference, fractured or loose pacemaker wires, etc.).

In some examples, the trigger event may include detection of an alert, notification, and/or instruction generated by the medical device 106(1). For example, the medical device may output an alert (e.g., via the GUI 128 and/or an aural alert) indicating a life-threating condition of the subject 102 (e.g., an alert indicating critical arrhythmia is detected, blood oxygen level is critically low, etc.), an alert indicating an error, a status, or an operation of the medical device (e.g., “sensor disconnection,” “low battery warning”, “software update is available,” etc.). In this instance, in response to detecting the trigger event, the medical device may cause data to be recorded that indicates how a user responds to the alert, notification, and/or instruction generated by the medical device 106(1). In at least one example, the trigger event may include detection of a lose of a connection (e.g., a wireless or wired connection) and/or a regain of the connection.

In some examples, the trigger event may include detection that an error code is triggered during device operation. For example, an NIBP device can generate errors indicating various issues such as bad signals (e.g., indicating the NIBP device failed to obtain a valid physiological signal), overpressure (e.g., the pressure in the system has exceeded a safe pressure level), air leaks in a hose, cuff, or connections, blockages in the air hose, and/or NIBP module malfunctions. The NIBP device may generate an error code indicating a subject 102 is moving, which can affect accuracy of the received physiological signal. To provide another non-limiting example, a capnography device can generate errors indicating one or more of an issue with a sampling line or airway issue (e.g., caused by a disconnected tube, clogged tube, misaligned tube, etc.), a sensor calibration error, sensor connection error, power supply error (e.g., due to low battery or internal power regulation issue), a loss of a signal, an error indicating the software versions between the main unit and sensor is incompatible (e.g., different manufactures may have different variances in their sampling line capabilities that can alter an accuracy of waveforms and values that are generated), measurement errors, and the like.

In some examples, the trigger event may include detection of an indication that a feature in use is not expected to function as well as intended. For example, the medical device 106(1) may be an AED. The AED may analyze the subject's heart rhythm during CPR to determine if a shock is recommended (e.g., using a Shock Advisory System (SAS)). The AED may also determine, using accelerometer data, that the subject is moving (e.g., the subject 102 may experience agonal respiration, which can cause some movement). In this example, the AED may not be able to accurately analyze the subject's heart rhythm. In this case, the SAS is not expected to function as well as intended.

In some examples, the trigger event may include a determination that the medical device 106(1) is being used for a high-risk or error-prone procedure (e.g., a procedure associated with a high likelihood of complications). In some instances, the trigger event may include a determination that the medical device 106(1) is being used in manner not approved by a manufacturer or regulatory agency (e.g., an off-label use). To provide non-limiting examples, the trigger event may be a determination that two defibrillators are being used simultaneously for higher energy delivery (e.g., for a patient experiencing refractory ventricular fibrillation), detecting use of a monitor-defibrillator for conditions other than ventricular fibrillation or ventricular tachycardia without a pulse, detecting use of electrodes that are not compatible with a defibrillator (e.g., electrodes manufactured by a third-party), and the like. To provide another non-limiting example, the medical device 106(1) may determine that a user has attempted cardioversion of atrial fibrillation in which a ventricular arrhythmia occurs within a period of time of the procedure. For instance, the medical device 106(1) may identify internal logs and signal data to infer a synchronized cardioversion event, identify activation of a “synchronized cardioversion” mode, delivery of a shock synchronized with an R-wave, detect a shock energy value consistent with cardioversion protocol (e.g., between about 50 joules to about 200 joules), detect a pre-shock rhythm classified as atrial fibrillation (e.g., irregularly irregular rhythm with no P-waves and narrow QRS).

In some examples, the trigger event may include detection of data indicating large changes between successive data points (e.g., detecting a threshold deviation between successive data points). For example, the medical device 106(1) may detect data indicating that electrode contact is inconsistent or poor (e.g., electrode impedance data indicates a poor connection). In some examples, the trigger event may include detecting data indicating certain subject characteristics are observed (e.g., transthoracic impedance). This can occur when there are changes in physiological data that are too drastic to be physiological, suggesting a technical artifact or sensor issue. For example, a heart rate of the subject 102 may be detected to be 102 bpm at a first time and 179 bpm at a second time, where the second time is about 1-2 seconds after the first time. This doubling in the heart rate indicates that the change in heart rate is unlikely to be physiological, and may be caused by a motion artifact (e.g., the patient moving or coughing), an electrode issue (e.g., loose or detached wire or lead), signal dropout or noise, etc.

In some examples, the trigger event may include detection of an unexpected user interaction with the medical device 106(1). An unexpected user interaction may include detecting a threshold number of touch screen inputs on a location of a touch screen of the medical device 106(1). The medical device 106(1) may include a display 126 including a touch screen and touch sensors that are integrated within the touch screen. When a finger or stylus touches the touch screen, the finger or stylus disrupts the electrostatic field of the touch screen. The disruption is measured as a change in capacitance. The medical device 106(1) may be configured to detect a threshold number of user inputs (e.g., 3 or more) within a period of time (e.g., within 3 seconds). This may indicate that a display of the medical device is malfunctioning. For example, the display 126 may not be registering the touch screen inputs. Alternatively, the unexpected user interaction may indicate that the user may be feeling frustrated or anxious, selecting an improper portion of the touch screen because the user interface element is too small, and the like. As illustrated in FIG. 1, the medical device 106(1) receives an indication of selection of the setting(s) 132 user interface element multiple times within a threshold period of time, indicating a trigger event 138(1).

In some examples, the trigger event may include detection of one or more keywords and/or a key phrase (e.g., trigger event 138(2) via a microphone. A keyword(s) can be an expletive or excessive expletives, profanity, swear words, etc. indicating frustration, anxiety, confusion, anger, etc. For example, keyword(s) or phrases may include “this is not working,” “I am confused,” “I have no idea if I am using it right,” “I have had it with this device,” “why does it keep beeping at me,” “I have to restart it,” and the like. In some examples, the medical device 106(1) may detect a key phrase indicating urgency, danger, a serious condition of the subject, such as “we're losing him/her,” “patient is going into cardiac arrest,” “she/he is crashing,” “code blue,” “not responsive,” “unresponsive,” “no pulse,” “she/he is not breathing,” “start compressions,” “massive bleed,” “I do not like the look of this,” and the like. As illustrated in FIG. 1, a microphone associated with the medical device 106(1) may be configured to detect a keyword and/or a key phrase uttered by a user or rescuer. A microphone disposed on the medical device 106(1) may be configured to detect speech. As another example, the medical device 106(1) may be communicatively coupled to another device that has a microphone and may receive, from the device, audio signals indicating speech. The microphone may be configured to record data in an audio channel. The medical device 106(1) (or a component of the medical device) may be configured to convert the detected audio data into digital text using a voice-to-text algorithm that analyzes sound waves, identifies phonemes, and uses language models to determine the most likely word sequence based on context and grammar. The voice-to-text processed data can indicate the user 104 (e.g., device operator) uttered one or more keywords.

In some examples, the trigger event may include detection of a condition or event that is associated with previous complaints or concerns. Complaints or concerns may be received from users, patients, customers, manufactures, medical personnel, researchers, etc. To provide a non-limiting example, complaints may have been raised regarding implanted cardioverter defibrillator (ICD) devices showing issues in determining the timing of individual cardiac depolarizations, also referred to as sensing issues, such as under-sensing of VF, atrial under-sensing, and/or over-sensing in relation to SVT-VT discrimination, and/or other unique issues related to sensing subcutaneous electrograms.

In some examples, the trigger event may include a determination that the medical device 106(1) is communicatively coupled to another computing device. The other computing device may include a second medical device (of a same or different type than the first medical device), a user interface device such as a smartphone, tablet, laptop, etc. In some instances, the separate computing device may include a microphone configured to capture audio data. The medical device 106(1) may be configured to receive audio signals from the other computing device. The medical device 106(1) may process the audio signals (e.g., via a speech-to-text system) and determine the audio data includes a keyword. In response to detecting the keyword in the audio data, the medical device 106(1) may store the audio data in a recorded file.

In some examples, the trigger event may include a determination that a first setting of a first medical device and a second setting of a second medical device are incompatible. For example, the medical device 106(1) may be communicatively coupled to a second medical device (e.g., medical device 106(2)). The first medical device can be associated with (or set to) a first setting or mode of operation. The first medical device can receive data from the second medical device. The received data can include one or more of physiological signals (ECG signals, glucose levels, etc.), device status or metadata (e.g., device mode or configuration), control or command signals indicating changes in settings or mode of operation, patient data (e.g., patient demographics), alarm triggers, device fault log, and the like. The received data can indicate that the second medical device is associated with a second setting or mode of operation that is incompatible with a first setting associated with the first medical device. To provide a non-limiting example, a dialysis device may cause fluid to be removed from a subject at a first rate and an infusion pump device may simultaneously administer fluids to the subject at a second rate.

In some examples, the trigger event may include a detection of a combination of events. For example, the trigger event may include a detection of a refractory ventricular fibrillation and a keyword(s) from the user. In some examples, detecting a first event may alter a threshold associated with detecting a second event. For example, the medical device 106(1) may detect a first event including detecting a first number of keywords within a first period of time (e.g., two expletives within a 10 second window), followed by detecting a second event including detecting a threshold number of user inputs within a second period of time (e.g., three button presses of the same button within a three second time window). In this example, the threshold number of user inputs may be lowered in response to detecting the first event (e.g., three button presses instead of five button presses).

In some examples, multiple trigger events may be detected within a threshold period of time (or during a rescue event). The trigger events may be associated with a hierarchy indicating a degree of severity and/or importance of the event. In the case where multiple trigger events are detected, the medical device may be configured to prioritize recording data related to a trigger event associated with a life-threatening condition (e.g., prioritize recording of data during a synchronized cardioversion event). For example, the medical device may detect, at a first time, a first trigger event associated with detecting a threshold number of keywords. The medical device can assign the first trigger event a first priority level (e.g., on a scale between 1-10, etc.). The medical device can then detect, at a second time after the first time, a second trigger event associated with detecting a QRS complex indicating potentially life-threatening heart rhythm such as ventricular tachycardia. The medical device may assign a second priority level to the second trigger event that is higher than the first priority level.

In some examples, the trigger event may include detecting user input from a user indicating the user wants to record data. For example, a user 104 may interact with an input device, such as a button on the medical device 106(1) in order to initiate recording, storing, and/or transmitting of data. In some examples, the user 104 may select a user interface element on the GUI 128 (not shown) in order to initiate recording, storing, and/or transmitting of data.

In some examples, the trigger event may include receiving a communication signal from a remote computing device. For example, the medical device 106(1) may receive a signal from a server that manages medical devices. The remote computing device(s) may receive direction from various sources (e.g., medical director, medical device manufacturer, etc.).

In some examples, the medical device 106(1) may receive a communicational signal including instructions to alter a trigger event, such as to add an additional trigger event, change parameters of a trigger event (e.g., alter a sample rate, alter a threshold to initiate storing of data, etc.), remove a trigger event, etc. That is, a computing device may be used to remotely configure a list of trigger events that a medical device responds to. In this way, as new trigger events become of interest (e.g., such as when a new device issue is suspected), the medical device 106(1) can respond to the new trigger event. In some examples, the communication signal may prompt the medical device 106(1) to initiate a firmware download from an external device. In at least one example, a list of trigger events may be altered at the medical device 106(1) level.

In some examples, the medical device 106(1) may be configured to disable detection of triggering events upon detecting signals indicating that a simulator (rather than a subject 102) is connected to the medical device 106(1).

In at least one example, the medical device 106(1) may receive an indication to stop recording and/or storing data (e.g., as a second trigger event). For example, the medical device 106(1) may receive user input (e.g., a button press or indication of selection of a user interface element) to stop storing data. In some examples, the medical device 106(1) may determine a threshold period of time has passed since a trigger event was detected and stop storing data in a file. In some examples, the medical device 106(1) may stop storing data in a file upon detecting a keyword(s) or key phrase (e.g., “the patient is stable”).

In some instances, in response to the trigger event, the medical device 106(1) may record and store data that is not generally or traditionally recorded and/or stored by the medical device, but that may be helpful in understanding the circumstances surrounding the event later on. For example, the medical device 106(1) may record and/or store environmental operating conditions, such as ambient temperature, humidity, internal device temperature, scene audio and/or video, light intensity, electromagnetic radiation present and measurable by the medical device sensors, device movement or vibration sensed by an accelerometer disposed on the medical device, data indicating interaction of the user and a response of the medical device, presence and/or strength of wireless communications signals detected by the device (e.g. Bluetooth, Wi-Fi, cellular, etc.), data indicating presence state of accessories and/or disposable for the device, battery status and/or its recent history, data indicating presence of paired device and/or detected device(s) proximate the medical device 106(1) (e.g., medical device 106(2)), and the like.

In response to detecting the trigger event, the medical device 106(1) may record and store a portion of data generated and/or received in association with the trigger event. That is, though the medical device 106(1) is configured to continuously record data, in some instances, only the data that may be considered relevant may be stored (e.g., data determined to satisfy a probability or likelihood that the data is associated with an event of interest). The portion of data may be stored in a memory (e.g., memory 714) of the medical device 106(1) for a period of time. In some examples, the medical device 106(1) may be configured to record certain types of data at a higher fidelity (e.g., physiological data) while other types of data may be recorded at a lower fidelity (e.g., environmental data, such as ambient temperature or humidity data). In some examples, the medical device 106(1) may determine to record particular types of data at a higher or lower fidelity in response to determining a type of trigger event. A type of trigger event can include at least one of a medical device error (e.g., detecting a shockable rhythm when it is not present, misinterpreting an artifact, etc.) or a user error (e.g., not enabling a synchronization mode for cardioversion, improperly placing sensors, ignoring or silencing an alarm, etc.). In some instances, the medical device 106(1) may record display output (e.g., by generating a screen recording of the GUI 128) at a first sample rate in response to detecting a first type of trigger event (e.g., detecting utterance of a threshold number of keywords) and can record the display output at a second sample rate (e.g., higher than the first sample rate) in response to detecting a second type of trigger event (e.g., physiological parameter artifact).

The portion of data may be stored in a file (or recorded file 140). As used herein, the term “file” and its equivalents may refer to a collection of data stored in a single unit. In some cases, a file may be identified by a file name. The medical device 106(1) may generate a recorded file 140 containing data from one or more data channels. The portion of data may correspond to a first period of time before the trigger event was detected and/or a second period of time after the trigger event was detected. The recorded file 140 may include high-fidelity data and/or low-fidelity data. For example, the recorded file 140 may include a first portion of data that is recorded at or above a threshold sample rate and a second portion of the data may be recorded below the threshold sample rate.

In some examples, the medical device 106(1) may be configured to transmit (e.g., via a transceiver(s) 742) the portion of data to one or more external computing device(s) 144 via communication network(s) 142. The external computing device(s) 144 may be associated with a sever, database, cloud computing platform, etc. The external computing device(s) 144 may be associated with a medical device manufacturer, a computing device within a clinical environment (e.g., a nurse station or other central monitoring station within a hospital), and the like. In some examples, the external computing device(s) 144 may extract data from the recorded file 140 and gain insight into the use and performance of the medical device 106(1). For example, the recorded file 140 can provide insight into how user(s) interact with the medical device 106(1), such as button press events, audio commands provided by the user, user response to device display output, etc. The collected data in the recorded file 140 may provide insight into potential improvements for the medical device, therapy needs, and/or monitoring needs of medical devices. The recorded file 140 can help guide development and improvement of features in medical devices including which features may be retired due to lack of use, malfunctioning, errors, etc.

In some examples, prior to transmitting data (or generating a file to store the data in non-volatile memory outside of a circular buffer), the medical device 106(1) may be configured to de-identify privileged data. Privileged data may include information that is sensitive, confidential, and/or is protected by laws and regulations to ensure patient privacy and confidentiality. In some instances, privileged data may include information that can be used to derive the identity of a particular subject, user (e.g., rescuer, paramedic, etc.) and/or rescue event. For instance, privileged data may include patient-specific data as well as voices, faces, and other characteristics of people (e.g., rescuers, bystanders, etc.) captured from the rescue scene. Privileged data may include physiological parameter data, at least portions of captured audio data, at least portions of captured video data, and the like.

In at least one example, the privileged data is captured and used to generate a record associated with the subject 102, which may be stored. For example, when treatment is initiated utilizing a monitor-defibrillator, a patient record or an event log may be generated to document details of treatment and/or monitoring of the subject 102 and the subject's response to the treatment. The patient record may include one or more of a date, time, location (e.g., geographic region, country, state, address, geographic coordinates, elevation, humidity level and/or temperature of the environment during the event, etc.) when treatment is administered, patient identification, type of treatment (e.g., particular treatment administered, such as defibrillation, cardioversion, or monitoring parameters adjusted), response to treatment (e.g., observations or assessments of the subject's response to the treatment, changes in vital signs, heart rhythm, symptoms, etc.), information association with the user 104 (e.g., healthcare provider) involved in administering treatment, outcome or result of the treatment administered (e.g., whether the treatment was successful in restoring normal heart rhythm or stabilizing a condition of the patient), an adverse event (e.g., documentation of a complication associated with a treatment), and the like. The privileged data, for instance, includes at least a portion of audio and/or video data captured from the patient and/or an ambient environment. In some examples, the privileged data may include sensitive data of a rescuer or bystander in the vicinity of the patient. In some cases, the privileged data is temporarily stored in a circular buffer.

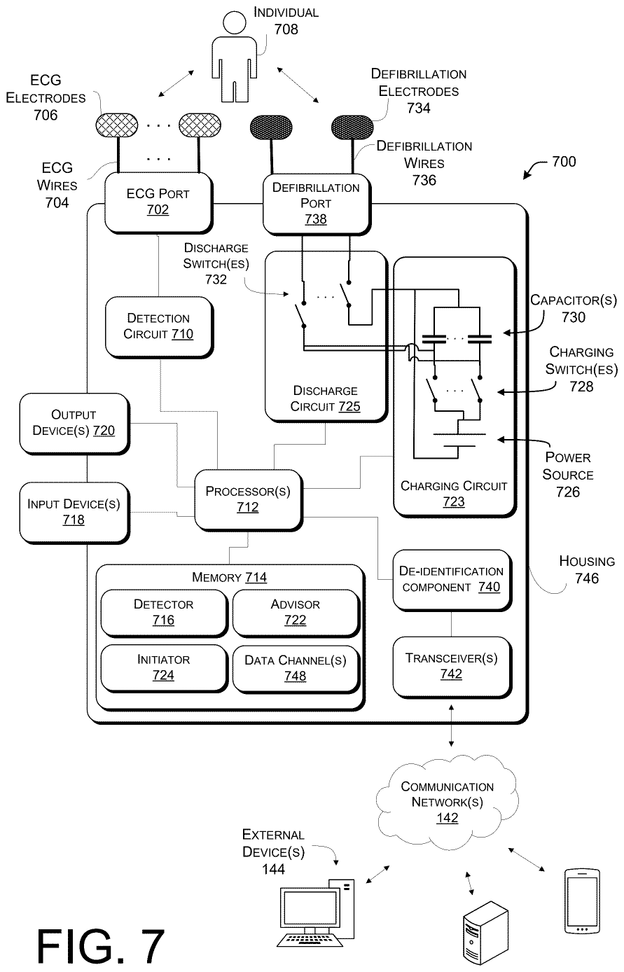

Transmission and storage of various types of privileged data described herein may be regulated or prohibited in view of various laws, rules, and regulations. For instance, the European Union (EU) enforces the General Data Protection Regulation (GDPR), governing the transfer of personal data collected and/or stored within the borders of EU member states. In some cases, entities controlling devices within EU borders are legally obligated, under the GDPR, to prevent personal data from being transferred to devices outside of EU borders. Various privacy laws are applicable to regions of the United States, including the California Consumer Privacy Act (CCPA), the Colorado Privacy Act, the Connecticut Data Privacy Act, the Delaware Personal Data Privacy Act, the Florida Data Privacy and Security Act, the Indiana Consumer Data Protection Act, the Iowa Consumer Data Protection Act, the Kentucky Consumer Data Protection Act, the Maryland Online Data Privacy Act, the Minnesota Consumer Data Privacy Act, the Montana Consumer Data Privacy Act, the Nebraska Data Privacy Act, the New Hampshire Consumer Expectation of Privacy Act, the New Jersey Personal Data Privacy Act, the Oregon Consumer Privacy Act, the Rhode Island Data Transparency and Privacy Protection Act, the Tennessee Information Protection Act, the Texas Data Privacy and Security Act, the Utah Consumer Privacy Act, and the Virginia Consumer Data Protection Act. Each of these laws specifies various types of privileged data. Moreover, at the Health Insurance Portability and Accountability Act (HIPAA) of 1996 specifies various types of health information that qualifies as privileged data, with various requirements for transmission and storage of such privileged data. Thus, it may be prohibited, in some cases, for the medical device 106(1) to transmit privileged data to an external device in violation of an applicable data privacy law that applies to the geographic region in which the medical device 106(1) or the external device is located. Nevertheless, other data generated by the medical device 106(1) may be highly pertinent to improving the medical device 106(1) or other medical devices, and/or identifying important trends associated with potential misuse of the medical device 106(1). De-identification of data can be performed by a de-identification component (e.g., de-identification component 740 discussed in relation to FIG. 7).

In some examples, a user (e.g., a researcher) may request to view the non-anonymized data in whole or in part. For example, a researcher may want to access a particular record associated with a subject in a non-anonymized format or a portion of a particular record. In some examples, the non-anonymized data and its anonymized counterpart may be tagged with the same identifier and stored at least temporarily in a memory on the medical device 106(1) and/or external memory (e.g., volatile memory (such as random-access memory (RAM)), and/or non-volatile memory (such as read only memory (ROM), flash memory, etc.).

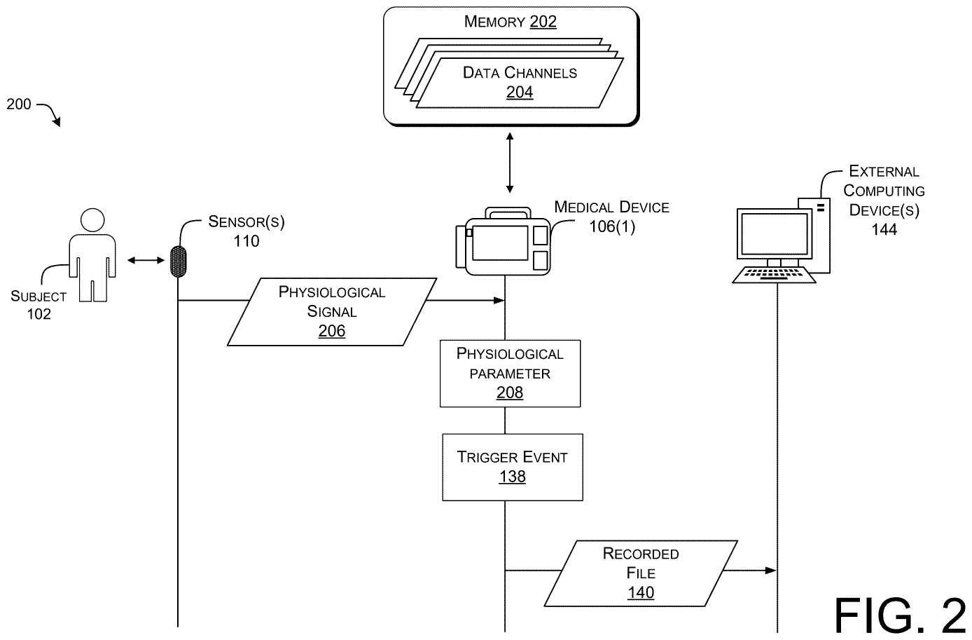

FIG. 2 illustrates example signaling 200 for facilitating communication between devices with reference to FIG. 1. In particular, the signaling 200 is between a sensor(s) 110, medical device 106(1), and external computing device(s) 144. Various messages within the signaling 200 are transmitted over at least one wired connection and/or at least one wireless connection. Although not specifically illustrated, various messages within the signaling 200 may be transmitted via one or more intermediary devices.

In examples, a sensor(s) 110 may be coupled to a subject 102 (e.g., a patient). The sensor 110 is configured to detect at least one physiological parameter of a subject 102. In various examples, the sensor(s) 110 includes at least one of an electrode, a detection circuit, a flow sensor, an oxygen sensor, a carbon dioxide sensor, a NIBP sensor (e.g., a blood pressure cuff, an ultrasound-based blood pressure sensor, etc.), an oxygenation sensor (e.g., a regional oximetry sensor, a pulse oximetry sensor), or the like. In some implementations the sensor(s) 110 is coupled to the medical device 106(1).

The sensor(s) 110 generates a physiological signal 206 (e.g., an analog signal, electrical signal, etc.). For example, an analog signal obtained from the sensor(s) 110 is converted into a digital format suitable for processing and analysis by a medical device's digital electronics. A physiological signal 206 is a signal that is generated based on a biological process or activity within a body of a subject 102. A physiological signal 206 can include various types of data, such as electrical, chemical, or thermal signals, which reflect different physiological processes.

The physiological signal 206 is detected and received by a medical device 106(1). The medical device 106(1) analyzes the physiological signal 206 in order to generate a physiological parameter 208 (e.g., a digital signal or data). A physiological parameter 208 can include, for example, vital signs (e.g., physiological parameters that reflect body functions such as body temperature, heart rate, blood pressure, respiratory rate, blood oxygenation (SpO₂)), cardiac parameters (e.g., parameters related to the cardiovascular system such as electrocardiogram (ECG/EKG), heart rhythm and heart rate variability, blood flow and cardiac output, etc.), respiratory parameters (e.g., end-tidal carbon dioxide (EtCO₂) levels, oxygen saturation, etc.), neurological parameters (e.g., electroencephalogram (EEG), intracranial pressure (ICP), neuromuscular function, etc.), metabolic parameters (e.g., blood glucose levels, blood pH, electrolyte levels, hormone levels, etc.), blood volume, blood flow, vascular resistance, and the like. In some instances, the medical device 106(1) may preprocess the physiological signal 206 to filter out noise, remove artifacts, and/or apply a signal processing algorithm to enhance a quality and/or accuracy of the physiological signal, such as frequency analysis, waveform analysis, or digital filtering techniques.

In some examples, multiple medical devices may communicate with one another and exchange treatment parameters (e.g., monitoring device, treatment device, receiving device, etc.). As used herein, the term “treatment parameter” and its equivalents, refers to a characteristic of a treatment performed on a subject 102. In at least one example, a first device (e.g., a treatment device) may be a chest compression device that reports a frequency of chest compressions administered by the first device to a second device (e.g., a monitoring device or receiving device). A receiving device may perform one or more actions based on the physiological parameter 208 and/or treatment parameter. Actions performed by a monitoring device or a treatment device include initiating a measurement of a physiological parameter 208 at a particular time or frequency, outputting a signal to a user, outputting a signal to the subject 102, performing a treatment at a particular time or frequency, adjusting a treatment parameter of an ongoing treatment, or any combination thereof. According to some examples, a monitoring device or a treatment device can instruct the other device to perform one or more actions. The receiving device, in turn, performs the action(s) based on the instruction from the monitoring device or the treatment device. For example, the monitoring device administers a defibrillation shock to the subject 102 at a particular time. By exchanging data or instructions, the monitoring device and the treatment device can coordinate monitoring and treatment of the subject 102.

To exchange data, multiple devices (e.g., a monitoring device and/or the treatment device) are configured to establish and/or communicate via a communication channel. As used herein, the term “communication channel,” and its equivalents, may refer to a medium over which a first endpoint (e.g., a sender) transmits information to one or more second endpoints (e.g., receivers). Examples of communication channels include wired connections, such as Ethernet or fiber optic paths, as well as wireless connections, such as Institute of Electronics and Electrical Engineers (IEEE) (e.g., WI-FI, BLUETOOTH, etc.) or 3^{rd} Generation Partnership Program (3GPP) (e.g., Long Term Evolution (LTE), New Radio (NR), etc.) connections. As used herein, the term “endpoint,” and its equivalents, may refer to an entity that is configured to transmit and/or receive data. Examples of endpoints include user equipment (UE) (e.g., mobile phones, tablet computers, etc.), computers, base stations, access points (APs), servers, compute nodes, medical devices, Internet of Things (IoT) devices, and the like.

In some implementations, the communication channel between multiple devices is established when the multiple devices are paired. In some examples, multiple devices may exchange one or more instructions, which can include an instruction to measure a physiological parameter, to begin a treatment, to end a treatment, a particular time or frequency at which to perform an action, an instruction to power on, an instruction to power off, an instruction to disconnect from a patient being treated or monitored, or a combination thereof. In particular cases, the multiple devices refrain from sharing substantive data (e.g., physiological parameters or other metrics, reports about the subject 102, instructions for treating the subject 102, etc.) until the devices are paired. As used herein, the term “paired,” and its equivalents, may refer to a state of multiple devices that have a shared link key that enables each device to cryptographically authenticate data it receives from any other device among the multiple devices. In some cases, paired devices communicate wirelessly.

The medical device 106(1) may detect a trigger event 138 in response to generating the physiological parameter 208. For example, the medical device 106(1) may detect a physiological parameter artifact (or a threshold number of artifacts). A physiological parameter artifact may include, for example, an ECG artifact (e.g., an electrode motion artifact causing signal dropout, a CPR artifact caused by chest compressions that mimic cardiac activity), a pulse oximetry artifact (e.g., an optical signal interference caused by ambient or bright light interfering with the pulse oximeter's sensor), capnography artifact (e.g., cardiac oscillations due to faster response time in children and neonates), and the like.

In response to detecting the trigger event 138, the medical device 106(1) may store data from one or more data channels 204 of the medical device 106(1). The data may represent a portion of the data the medical device 106(1) is actively recording. The portion of data may be stored in a memory 202 of the medical device 106(1) (e.g., for a period of time). The portion of data stored can be associated with the trigger event 138. The medical device 106(1) can generate and store the portion of data in a recorded file 140. The recorded file 140 may include high-fidelity data and/or low-fidelity data.

The medical device 106(1) transmits the recorded file 140 (e.g., by transmitting a communication signal(s) such as analog signals, cellular transmission, etc.) to an external computing device(s) 144. In some examples, the external computing device(s) 144 may analyze the recorded file 140 in order identify information related to an event of interest or an anomaly, identify misuse of a medical device 106(1), etc. In some examples, the external computing device(s) 144 may receive multiple recorded files associated with the same rescue event (e.g., from multiple devices present at the rescue scene) and can store the multiple recorded files together in a database.

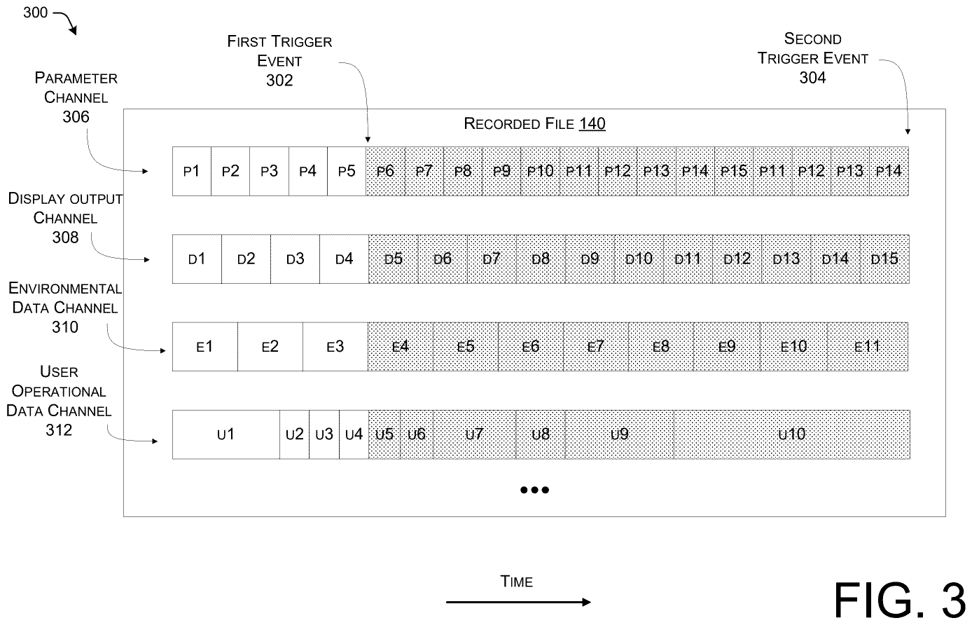

FIG. 3 illustrates an example of a recorded file 140 described with reference to FIGS. 1 and 2. In FIG. 3, times increases from left to right. The illustrated recorded file 140 includes four channels, although the recorded file 140 can include more or less data channels and/or different types of data channels. The values in the channels can be detected by the medical device 106(1) at a particular sampling frequency, such as once a minute, five times a minute, 10 times a minute, 30 times a minute, once a second, or multiple times a second. That is, the values in the channel(s) can be sampled at discrete (sampling) times. The values are the sampled values. Although various channels and types of values or parameters are possible in various implementations in the present disclosure, FIG. 3 illustrates four channels including a parameter channel 306, a display output channel 308, an environmental data channel 310, and a user operational data channel 312.

Individual channels can be configured to record different types of data. For example, the parameter channel 306 can include physiological parameters. The display output channel 308 can include video stream data capturing visual content rendered on the user interface of the medical device 106(1). The display output channel 308 can include a screen recording that illustrates what was presented to a user during a period of time associated with a trigger event (e.g., 5 second before a trigger event was detected and 25 seconds after the trigger event was detected). The screen recording may be captured at or above a threshold sample rate (e.g., at or above 10 kilohertz). In at least one example, the threshold sample rate is at or above 40 kilohertz. The screen recording may be captured at or above a threshold frame rate (e.g., at or above 30 frames per second (fps)). The screen recording may be captured at or above a resolution (e.g., 720p, 1080p, etc.).

The environmental data channel 310 can include external environment data indicating a state of the environment surround the medical device 106(1) (e.g., ambient temperature, humidity, ambient light intensity, etc.). The sampling frequency of the environmental data may be recorded at regular or irregular time intervals. For example, every second, minute, etc. Alternatively, the environmental data can be recorded once upon detecting a trigger event.

The user operational data channel 312 can include user input signals detected by the medical device 106(1). User input data can include data indicating button presses (mechanical or capacitive), knob or dial rotations, switch toggles, slider input, touch screen taps or swipes, keyboard input, mouse input (or pointer-based interactions), physical attachments such as inserting a probe or attaching a sensor, pugging in a cable or other connector, and the like. In some instances, user input data can include voice commands or keywords detected by a microphone (e.g., a microphone disposed on the medical device or via a microphone that is communicatively coupled to the medical device). In some examples, different types of user input data can be recorded in separate channels or sub-channels (e.g., button presses may be stored in a first channel and voice commands can be stored in a second channel).

The recorded file 140 may include timestamps. A timestamp can indicate when a trigger event was detected (e.g., first trigger event 302). In some examples, various data types within channels can be aligned by time (i.e., time-aligned). The medical device 106(1) may detect a second trigger event 304 indicating the medical device 106(1) stopped recording data and/or storing the data in the file. For example, the medical device 106(1) can stop storing data in the file 140 after a predetermined period of time following the first trigger event 302, upon determining a threshold amount of data has been stored in the recorded file 140, after detecting a user input, and the like.

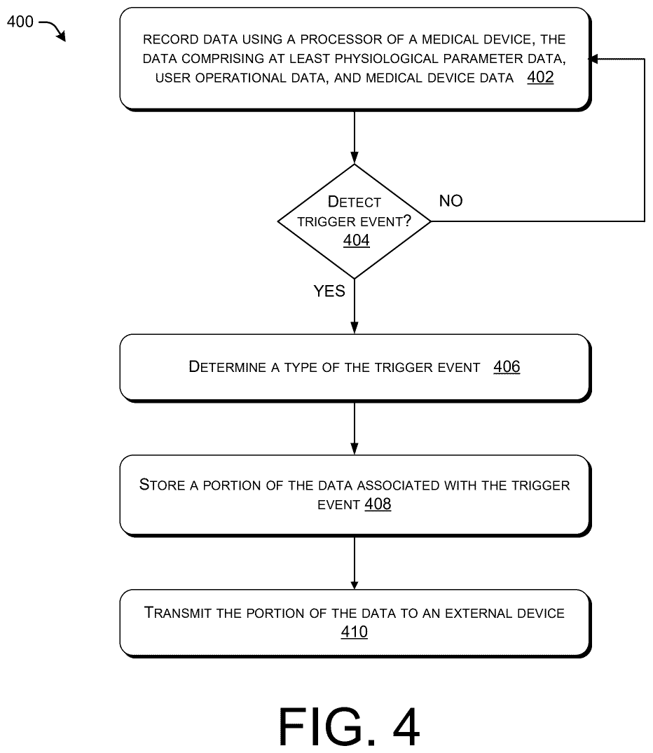

FIG. 4 illustrates an example process 400 for recording data, detecting a trigger event, storing a portion of the recorded data, and transmitting the portion of the recorded data to an external device. The example process 400 can be performed by an entity, such as a processor or other component of a medical device.

At 402, the entity of the medical device continuously records data in a circular buffer. The data can include parameter data indicating a physiological parameter of a subject. For example, the parameter data includes discrete physiological parameter measurements detected at discrete times, wherein the discrete times corresponding to a threshold sample rate of the medical device. The parameter data indicates the physiological parameter measurements in a first channel (e.g., a “physiological parameter channel”). In some cases, the data includes user operational data indicating user input signals detected by the medical device. The data may include medical device data indicating output signals generated by the medical device. Medical device data can include, for example, data generated and/or received by the medical device, such as data that is displayed on the display screen, device status data indicating a state of the medical device, and the like. Medical device data can indicate a battery level, an operational mode the medical device is in (e.g., standby or active), firmware/software version of the medical device, component status (e.g., sensor connected/disconnected), error codes or fault logs, diagnostic data, configuration and/or setting data (e.g., alarm thresholds, measurement intervals, etc.), self-test log(s) and/or calibration results, communication and connectivity data (e.g., network status, Bluetooth/Wi-Fi, pairing logs, etc.), and the like. In some cases, the circular buffer is part of short-term storage (e.g., volatile storage) within memory of the entity.

At 404, the entity determines whether a trigger event is detected. If at block 404, the entity does not detect a trigger event, the process may follow the “NO” route and proceeds back to block 420. If at block 404, the entity detects a trigger event, the process follows the “YES” route and proceeds to block 406.

At 406, the entity determines a type of trigger event. A type of trigger event can include a medical device error (e.g., detecting a shockable rhythm when it is not present, detecting an artifact, etc.) or a user error (e.g., not enabling a synchronization mode for cardioversion, detecting improper placement of sensors, determining an alarm or alert was ignored, etc.). In some instances, the entity generates a screen recording of the display screen at a first sample rate in response to detecting a first type of trigger event (e.g., detecting utterance of a threshold number of keywords) and generates the screen recording at a second sample rate (e.g., higher than the first sample rate) in response to detecting a second type of trigger event (e.g., physiological parameter artifact).

At 408, the entity stores a portion of the recorded data associated with the trigger event. The portion of data may be stored in a file (e.g., recorded file 140). The file may contain data from one or more data channels. The portion of data may correspond to a first period of time before the trigger event was detected and a second period of time after the trigger event was detected. The file can include high-fidelity data and low-fidelity data. For example, the file may include parameter data that is recorded at or above a threshold sample rate and environmental data recorded below the threshold sample rate. The file is stored outside of the circular buffer, for instance. In some cases, the file is stored in long-term storage (e.g., non-volatile storage) within the memory.

At 410, the entity transmits the potion of the data to an external device. For example, the entity transmits a file containing data associated with the trigger event over a wireless and/or wired interface. In some instances, the entity transmits the portion of data to the external device automatically (i.e., without requiring user input confirming to send the portion of data).

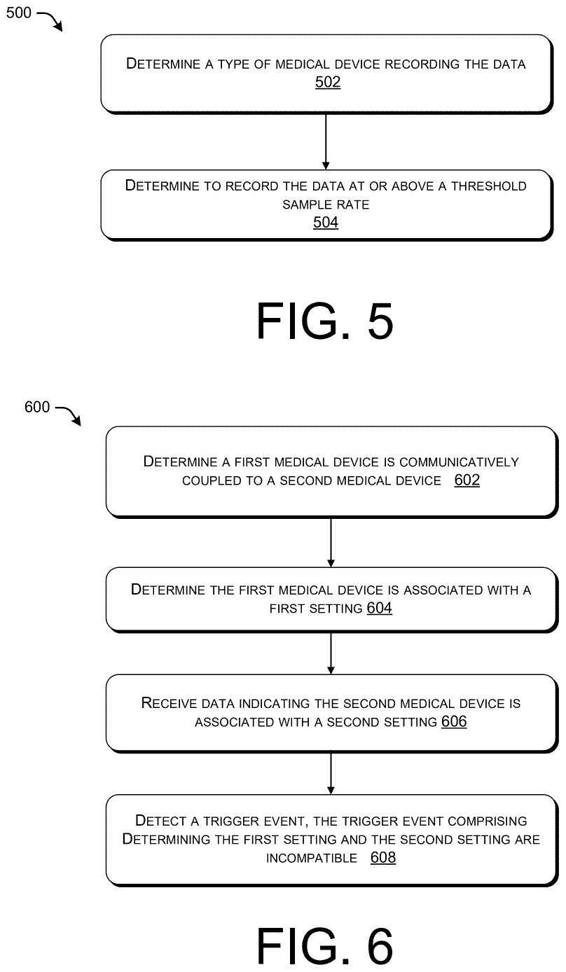

FIG. 5 illustrates an example process 500 for determining a type of medical device that is recording data and determining to record the data at or above a threshold sample rate. The example process 500 can be performed by an entity, such as a processor or other component of a medical device.

At 502, the entity determines a type of medical device recording the data. For example, a type of medical device can include a monitoring device (e.g., patient monitor, ECG machine, blood glucose monitor, pulse oximeter, etc.), life-support device used to treat, support, or maintain critical body functions (e.g., defibrillator (manual or AED), pacemaker, implantable cardioverter-defibrillator, ventilator, infusion pump, dialysis machine, etc.), diagnostic device (e.g., ultrasound machine, MRI scanner, X-ray machine, CT scanner, etc.), and the like.

At 504, the entity determines to record data at or above a threshold sample rate in response to determining the type of medical device. For example, if the medical device is a pulse oximeter, the entity can record oxygen saturation data at a sample rate of between about 1 Hz (1 sample per second) and record oximeter waveform data at a sample rate of between about 30 Hz to about 200 Hz. Alternatively, if the medical device measures ECG parameters, the sample rate between about 125 Hz and about 500 Hz. In at least one example, the file includes a recorded MP3 file of audio data of an environment surrounding the medical device, the MP3 file having a sample rate of about 48 kHz.

FIG. 6 illustrates an example process for detecting a trigger event indicating a first setting associated with a first medical device that is communicatively coupled to a second medical device is incompatible with a second setting associated with the second medical device. The example process 600 can be performed by an entity, such as a processor or other component of a medical device.

At 602, the entity determines a first medical device is communicatively coupled to a second medical device 602. The first medical device can be communicatively paired to the second medical device via a wireless or wired interface.

At 604 the entity determines the first medical device is associated with a first setting or mode of operation. For example, a user may change a setting on the first medical device, or the medical device can default to a particular setting or mode (e.g. adult mode in a monitor-defibrillator). A display screen of a medical device may present various mode options or indications. The mode indications indicate filters or algorithms that are active. In some examples, the mode indication presented is selectable, such that a user can activate and/or deactivate various modes (e.g., advisory mode, manual mode, adult mode, pediatric mode, etc.).

At 606, the entity receives data from the second medical device indicating the second medical device is associated with a second setting. For example, the first medical device may receive data can including one or more of physiological parameter signals, device status or metadata (e.g., device mode or configuration), control or command signals indicating changes in settings or mode of operation, patient data (e.g., patient demographics), alarm triggers, device fault log, model number, a number of leads connected to the medical device, a type of lead connected to the medical device, a battery life, a volume level, a condition of one or more accessories associated with the medical device, physiological parameter data, shock advisory data, audio output data, display output data, or device motion during operation of the medical device, and the like. The received data can indicate that the second medical device is associated with a second setting or mode of operation that is incompatible with a first setting associated with the first medical device.

At 608, the entity can detect a trigger event based on determining that the first setting and the second setting are incompatible. For example, if audio CPR prompts for a monitor/defibrillator and a chest compression device are setup in a way that conflict with each other, the entity can detect the conflict in settings and initiate storing of recorded data in a file. Detecting a trigger event can prompt the entity to store a portion of the recorded data in a file. The entity can transmit the file to an external device. In some instances, the entity may generate and transmit the file in response to an event (e.g., after a threshold period of time, in response to recording a threshold quantity of data associated with the trigger event, in response to detecting an end of a rescue event, etc.).

FIG. 7 illustrates an example external defibrillator 700 configured to perform various functions described herein. For example, the external defibrillator 700 is the medical device 106(1) described above with reference to FIG. 1.

The external defibrillator 700 includes an electrocardiogram (ECG) port 702 connected to multiple ECG wires 704. In some cases, the ECG wires 704 are removeable from the ECG port 702. For instance, the ECG wires 704 are plugged into the ECG port 702 via connectors. The ECG wires 704 are connected to ECG electrodes 706, respectively. In various implementations, the ECG electrodes 706 are disposed on different locations on an individual 708. A detection circuit 710 is configured to detect relative voltages between the ECG electrodes 706. These voltages are indicative of the electrical activity of the heart of the individual 708.

In various implementations, the ECG electrodes 706 are in contact with the different locations on the skin of the individual 708. In some examples, a first one of the ECG electrodes 706 is placed on the skin between the heart and right arm of the individual 708, a second one of the ECG electrodes 706 is placed on the skin between the heart and left arm of the individual 708, and a third one of the ECG electrodes 706 is placed on the skin between the heart and a leg (either the left leg or the right leg) of the individual 708. In these examples, the detection circuit 710 is configured to measure the relative voltages between the first, second, and third ECG electrodes 706. Respective pairings of the ECG electrodes 706 are referred to as “leads,” and the voltages between the pairs of ECG electrodes 706 are known as “lead voltages.” In some examples, more than three ECG electrodes 706 are included, such that 5-lead or 12-lead ECG signals are detected by the detection circuit 710.

The detection circuit 710 includes at least one analog circuit, at least one digital circuit, or a combination thereof. The detection circuit 710 receives the analog electrical signals from the ECG electrodes 706, via the ECG port 702 and the ECG wires 704. In some cases, the detection circuit 710 includes one or more analog filters configured to filter noise and/or artifacts from the electrical signals. The detection circuit 710 includes an analog-to-digital (ADC) in various examples. The detection circuit 710 generates a digital signal indicative of the analog electrical signals from the ECG electrodes 706. This digital signal can be referred to as an “ECG signal” or an “ECG.”

In some cases, the detection circuit 710 further detects an electrical impedance between at least one pair of the ECG electrodes 706. For example, the detection circuit 710 includes, or otherwise controls, a power source that applies a known voltage (or current) across a pair of the ECG electrodes 706 and detects a resultant current (or voltage) between the pair of the ECG electrodes 706. The impedance is generated based on the applied signal (voltage or current) and the resultant signal (current or voltage). In various cases, the impedance corresponds to respiration of the individual 708, chest compressions performed on the individual 708, and other physiological states of the individual 708. In various examples, the detection circuit 710 includes one or more analog filters configured to filter noise and/or artifact from the resultant signal. The detection circuit 710 generates a digital signal indicative of the impedance using an ADC. This digital signal can be referred to as an “impedance signal” or an “impedance.”

The detection circuit 710 provides the ECG signal and/or the impedance signal one or more processors 712 in the external defibrillator 700. In some implementations, the processor(s) 712 includes a central processing unit (CPU), a graphics processing unit (GPU), both CPU and GPU, or other processing unit or component known in the art.

The processor(s) 712 is operably connected to memory 714. In various implementations, the memory 714 is volatile (such as random-access memory (RAM)), non-volatile (such as read only memory (ROM), flash memory, etc.) or some combination of the two. The memory 714 stores instructions that, when executed by the processor(s) 712, causes the processor(s) 712 to perform various operations. In various examples, the memory 714 stores methods, threads, processes, applications, objects, modules, any other sort of executable instruction, or a combination thereof. In some cases, the memory 714 stores files (e.g., recorded file 140), databases, or a combination thereof. In some examples, the memory 714 includes, but is not limited to, RAM, ROM, electrically erasable programmable read-only memory (EEPROM), flash memory, or any other memory technology. In some examples, the memory 714 includes one or more of CD-ROMs, digital versatile discs (DVDs), content-addressable memory (CAM), or other optical storage, magnetic cassettes, magnetic tape, magnetic disk storage or other magnetic storage devices, or any other medium which can be used to store the desired information and which can be accessed by the processor(s) 712 and/or the external defibrillator 700. In some cases, the memory 714 at least temporarily stores the ECG signal and/or the impedance signal.

The processor(s) 712 is operably connected to one or more input devices 718 and one or more output devices 720. Collectively, the input device(s) 718 and the output device(s) 720 function as an interface between a user and the defibrillator 700. The input device(s) 718 is configured to receive an input from a user and includes at least one of a keypad, a cursor control, a touch-sensitive display, a voice input device (e.g., a microphone), a haptic feedback device (e.g., a gyroscope), or any combination thereof. The output device(s) 720 includes at least one of a display, a speaker, a haptic output device, a printer, or any combination thereof. In various examples, the processor(s) 712 causes a display among the input device(s) 718 to visually output a waveform of the ECG signal and/or the impedance signal. In some implementations, the input device(s) 718 includes one or more touch sensors, the output device(s) 720 includes a display screen, and the touch sensor(s) are integrated with the display screen. Thus, in some cases, the external defibrillator 700 includes a touchscreen configured to receive user input signal(s) and visually output physiological parameters, such as the ECG signal and/or the impedance signal.

In various examples, the memory 714 includes a detector 716, which causes the processor(s) 712 to determine, based on the ECG signal and/or the impedance signal, whether the individual 708 is exhibiting a particular heart rhythm. For instance, the processor(s) 712 determines whether the individual 708 is experiencing a shockable rhythm that is treatable by defibrillation. Examples of shockable rhythms include ventricular fibrillation (VF) and ventricular tachycardia (VT). In some examples, the processor(s) 712 determines whether any of a variety of different rhythms (e.g., asystole, sinus rhythm, atrial fibrillation (AF), etc.) are present in the ECG signal.

In some examples, the memory 714 includes an advisor 722, which, when executed by the processor(s) 712, causes the processor(s) 712 to generate advice and/or control the output device(s) 720 to output the advice to a user (e.g., a rescuer) via an output device (e.g., via a message or alert on the display screen). In some examples, the processor(s) 712 provides, or causes the output device(s) 720 to provide, an instruction to perform CPR on the individual 708. In some cases, the processor(s) 712 evaluates, based on the ECG signal, the impedance signal, or other physiological parameters, CPR being performed on the individual 708 and causes the output device(s) 720 to provide feedback about the CPR in the instruction. According to some examples, the processor(s) 712, upon identifying that a shockable rhythm is present in the ECG signal, causes the output device(s) 720 to output an instruction and/or recommendation to administer a defibrillation shock to the individual 708.

The memory 714 also includes an initiator 724 which, when executed by the processor(s) 712, causes the processor(s) 712 to control other elements of the external defibrillator 700 in order to administer a defibrillation shock to the individual 708. In some examples, the processor(s) 712 executing the initiator 724 selectively causes the administration of the defibrillation shock based on determining that the individual 708 is exhibiting the shockable rhythm and/or based on an input from a user (received, e.g., by the input device(s) 718). In some cases, the processor(s) 712 causes the defibrillation shock to be output at a particular time, which is determined by the processor(s) 712 based on the ECG signal and/or the impedance signal.

The memory 714 includes data channel(s) 748. Data channels can be logical streams or pathways that can collect, transmit, or store particular types of data. Data channels can include data packets. Data channels can include data received from a variety of sources(e.g., sensor, processor, user interaction, external device, etc.). Channels can have different formats depending on the type of data it is associated with (e.g., waveform, text, numeric, etc.). A data channel can be associated with a sampling rate defining how often data is recorded in the channel. For example, a data channel can include a parameter data channel including physiological signals recorded at one or more sample rates (or within a range of sample rates). Data channel(s) can include an event data channel including data indicating an alarm was triggered (e.g., detecting a heart rate is above at or above a threshold heart rate), a manual event (e.g., user pressed a button), system event (e.g., battery level low).

The external defibrillator 700 includes a de-identification component 740 configured to generate de-identified data. De-identification of data may include removing and/or obscuring identifying data such that patient, rescuer, and/or bystander privacy is protected while still enabling the data to be utilized for research, analysis, or other purposes, as discussed above in relation in FIG. 1. For example, the de-identification component 740 may anonymize identifying data (e.g., by removing direct identifiers, such as names, addresses, medical identification numbers, voices in audio, faces in video, and/or any other information that could directly identify a subject or other person in the vicinity of the external defibrillator 700 during a rescue event), pseudonymize identifying data (e.g., replace identifying information with artificial identifiers or pseudonyms such that the data is not directly linked to the identity of a subject or other person), generalize the identifying data (e.g., generalize or aggregate data to a broader level such as an age range), suppress the identifying data (e.g., by removing or suppressing a data field from a dataset that meets a threshold), mask the data (e.g., by replacing particular data values with similar but fictitious values), tokenize the identifying data (e.g., by replacing the identifying data with randomly generalized tokens or codes that are utilized to represent the original data without revealing the identifying data), and the like. In some instances, a combination of de-identification techniques may be utilized to de-identify different types of data. In some instances, the de-identification component 740 can be configured to generate de-identified data based at least in part on encrypting, via a processor, the privileged data using an encryption algorithm and a unique encryption key prior to transmitting the data to an external device via transceiver 742.

In some instances, the de-identification component 740 may be configured to generate de-identified data based at least in part on encrypting, via a processor, the privileged data using an encryption algorithm and a unique encryption key prior to transmitting the data to an external device. As used herein, the term “encrypt,” and its equivalents, refers to a process of translating data from one format (e.g., an unencoded format) into an encoded format. In various cases, the encoded format is referred to as “ciphertext.” Unencoded data, which has not been encrypted, may be referred to as being in “plaintext.” In various examples, a de-identification component associated with the medical device encrypts data using at least one encryption key. An encryption key is a parameter that defines the translation of data from the one format into the encoded format. As used herein, the term “decrypt,” and its equivalents, refers to a process of translating data from an encoded format into another format (e.g., an unencoded format), such as a plaintext format. In various examples, an entity encrypts data using at least one decryption key. A decryption key is a parameter that defines the translation of data from the encoded format into the other format. A link key, for example, is an encryption and/or decryption key.

Various cryptographic techniques can be utilized in accordance with the features described in this disclosure. For example, data can be encrypted and decrypted via a symmetric key, wherein the encryption key and the decryption key are equivalent. In some cases, data can be encrypted and decrypted via asymmetric keys, wherein the encryption key and the decryption key are different. Cryptographic hash functions (CHFs) are examples of cryptographic techniques. Examples of cryptographic techniques include the Data Encryption Standard (DES), Advanced Encryption Standard (AES), Elliptic Curve Cryptography (ECC), Rivest-Shamir-Adleman (RSA), Secure Hash Algorithm (SHA)-1, SHA-2, SHA-3, BLAKE, BLAKE2, BLAKE3, WHIRLPOOL, MD2, MD4, MD5, MD6, Temporal Key Integrity Protocol (TKIP), Rivest cipher 4 (RC4), variably modified permutation composition (VMPC), blowfish, Twofish, Threefish, Tiny Encryption Algorithm (TEA), Extended TEA (XTEA), Corrected Block TEA (XXTEA), Diffie-Hellman exchange (DHE), elliptic curve DHE, super-singular isogeny Diffie-Hellman (SIDH) key exchange, and so on. Any suitable encryption or decryption technique can be used in accordance with implementations of this disclosure.

The processor(s) 712 is operably connected to a charging circuit 723 and a discharge circuit 725. In various implementations, the charging circuit 723 includes a power source 726, one or more charging switches 728, and one or more capacitors 730. The power source 726 includes, for instance, a battery. The processor(s) 712 initiates a defibrillation shock by causing the power source 726 to charge at least one capacitor among the capacitor(s) 730. For example, the processor(s) 712 activates at least one of the charging switch(es) 728 in the charging circuit 723 to complete a first circuit connecting the power source 726 and the capacitor to be charged. Then, the processor(s) 712 causes the discharge circuit 725 to discharge energy stored in the charged capacitor across a pair of defibrillation electrodes 734, which are in contact with the individual 708. For example, the processor(s) 712 deactivates the charging switch(es) 728 completing the first circuit between the capacitor(s) 730 and the power source 726 and activates one or more discharge switches 732 completing a second circuit connecting the charged capacitor 730 and at least a portion of the individual 708 disposed between defibrillation electrodes 734.

The energy is discharged from the defibrillation electrodes 734 in the form of a defibrillation shock. For example, the defibrillation electrodes 734 are connected to the skin of the individual 708 and located at positions on different sides of the heart of the individual 708, such that the defibrillation shock is applied across the heart of the individual 708. The defibrillation shock, in various examples, depolarizes a significant number of heart cells in a short amount of time. The defibrillation shock, for example, interrupts the propagation of the shockable rhythm (e.g., VF or VT) through the heart. In some examples, the defibrillation shock is 200 J or greater with a duration of about 0.015 seconds. In some cases, the defibrillation shock has a multiphasic (e.g., biphasic) waveform. The discharge switch(es) 732 are controlled by the processor(s) 712, for example. In various implementations, the defibrillation electrodes 734 are connected to defibrillation wires 736. The defibrillation wires 736 are connected to a defibrillation port 738, in implementations. According to various examples, the defibrillation wires 736 are removable from the defibrillation port 738. For example, the defibrillation wires 736 are plugged into the defibrillation port 738.

In various implementations, the processor(s) 712 is operably connected to one or more transceivers 742 that transmit and/or receive data over one or more communication networks 142. For example, the transceiver(s) 742 includes a network interface card (NIC), a network adapter, a local area network (LAN) adapter, or a physical, virtual, or logical address to connect to the various external devices and/or systems. In various examples, the transceiver(s) 742 includes any sort of wireless transceivers capable of engaging in wireless communication (e.g., radio frequency (RF) communication). For example, the communication network(s) 142 includes one or more wireless networks that include a 3^{rd} Generation Partnership Project (3GPP) network, such as a Long Term Evolution (LTE) radio access network (RAN) (e.g., over one or more LTE bands), a New Radio (NR) RAN (e.g., over one or more NR bands), or a combination thereof. In some cases, the transceiver(s) 742 includes other wireless modems, such as a modem for engaging in WI-FI®, WIGIG®, WIMAX®, BLUETOOTH®, or infrared communication over the communication network(s) 142.

In various implementations, the external defibrillator 700 also includes a housing 746 that at least partially encloses other elements of the external defibrillator 700. For example, the housing 746 encloses the detection circuit 710, the processor(s) 712, the memory 714, the charging circuit 723, the de-identification component 740, the transceiver(s) 742, or any combination thereof. In some cases, the input device(s) 718 and output device(s) 720 extend from an interior space at least partially surrounded by the housing 746 through a wall of the housing 746. In various examples, the housing 746 acts as a barrier to moisture, electrical interference, and/or dust, thereby protecting various components in the external defibrillator 700 from damage.

In some implementations, the external defibrillator 700 is an automated external defibrillator (AED) operated by an untrained user (e.g., a bystander, layperson, etc.) and can be operated in an automatic mode. In automatic mode, the processor(s) 712 automatically identifies a rhythm in the ECG signal, makes a decision whether to administer a defibrillation shock, charges the capacitor(s) 730, discharges the capacitor(s) 730, or any combination thereof. In some cases, the processor(s) 712 controls the output device(s) 720 to output (e.g., display) a simplified user interface to the untrained user. For example, the processor(s) 712 refrains from causing the output device(s) 720 to display a waveform of the ECG signal and/or the impedance signal to the untrained user, in order to simplify operation of the external defibrillator 700.

In some examples, the external defibrillator 700 is a monitor-defibrillator utilized by a trained user (e.g., a clinician, an emergency responder, etc.) and can be operated in a manual mode or the automatic mode. When the external defibrillator 700 operates in manual mode, the processor(s) 712 cause the output device(s) 720 to display a variety of information that may be relevant to the trained user, such as waveforms indicating the ECG data and/or impedance data, notifications about detected heart rhythms, and the like.

The external defibrillator 700 is configured to transmit and/or receive data (e.g., ECG data, impedance data, data indicative of one or more detected heart rhythms of the individual 708, data indicative of one or more defibrillation shocks administered to the individual 708, etc.) with one or more external devices 144 via the communication network(s) 142. The external devices 144 include, for instance, mobile devices (e.g., mobile phones, smart watches, etc.), Internet of Things (IoT) devices, medical devices, computers (e.g., laptop devices, servers, etc.), or any other type of computing device configured to communicate over the communication network(s) 142. In some examples, the external device(s) 144 is located remotely from the external defibrillator 700, such as at a remote clinical environment (e.g., a hospital), research facility, a manufacturing facility associated with the external defibrillator 700, etc. According to various implementations, the processor(s) 712 causes the transceiver(s) 742 to transmit data to the external device(s) 144 associated with a customer system(s) (e.g., hospital(s) subscribed to a data service that provides the hospital access to the data). In some instances, the customer system(s) may receive the de-identified data in an encrypted format as well as a decryption key that enables the customer system(s) to decrypt the de-identified data.

### Example Clauses

The following clauses provide various examples of the present disclosure:

1. A monitor-defibrillator including: a monitoring circuit configured to detect an electrical signal indicative of an electrocardiogram (ECG) of a subject during a rescue event; a parameter sensor configured to detect a physiological parameter of the subject; a display configured to output a visual representation of the ECG; an input device configured to receive an input signal from a user; a treatment circuit configured to output an electrical shock to a heart of the subject in response to the input device receiving the input signal from the user; memory; a transceiver; and a processor configured to: continuously record high-fidelity data including at least physiological parameter data, user operational data, and device data during the rescue event; detect a trigger event indicating an anomaly occurred during the rescue event; in response to detecting the trigger event, cause the memory to store a portion of the high-fidelity data corresponding to a period of time after the trigger event; and cause the transceiver to transmit the portion of the high-fidelity data to an external device.

2. The monitor-defibrillator of clause 1, wherein the high-fidelity data includes at least one: data recorded at or above a threshold sample rate; or data recorded at or above a threshold filtering bandwidth.

3. The monitor-defibrillator of clause 1 or 2, the portion being a first portion, the period of time being a first period of time, wherein the high-fidelity data further includes a second portion corresponding to a second period of time that occurs before detecting the trigger event, the processor further configured to: in response to detecting the trigger event, cause the memory to store the second portion of high-fidelity data corresponding to the second period of time; and cause the transceiver to transmit the second portion of the high-fidelity data to the external device.

4. The monitor-defibrillator of any of clauses 1 to 3, wherein the portion of the high-fidelity data includes display data indicating information visually presented on the display.

5. A medical device, including: a physiological sensor configured to detect a physiological parameter of a subject; an input device configured to detect an input signal from a user; memory; a transceiver; and a processor configured to: continuously record high-fidelity data including at least physiological parameter data, user operational data, and device data; detect a trigger event; in response to detecting the trigger event, cause the memory to store a portion of the high-fidelity data corresponding to a period of time after the trigger event; and cause the transceiver to transmit the portion of the high-fidelity data to an external device.

6. The medical device of clause 5, wherein the medical device includes a monitor-defibrillator, an automated external defibrillator (AED), a non-invasive blood pressure monitor (NIBP), a chest compression device, a patient monitor, a medical cot, an implantable cardioverter-defibrillator (ICD), or a pacemaker.

7. The medical device of clauses 5 or 6, the processor further configured to: determine a type of the medical device; and in response to determining the type of the medical device, determine a threshold sample rate of the high-fidelity data.

8. The medical device of any of clauses 5 to 7, wherein the physiological parameter includes an ECG, a capnograph, an end-tidal carbon dioxide (EtCO₂), an airway parameter, a blood oxygenation, a blood flow parameter, a blood pressure, a heart rate, or a temperature.

9. The medical device of any of clauses 5 to 8, wherein the user operational data includes user input signals detected by the medical device.

10. The medical device of any of clauses 5 to 9, wherein the device data includes output signals provided by the medical device.

11. The medical device of any of clauses 5 to 10, wherein the trigger event includes detecting a physiological parameter artifact.

12. The medical device of any of clauses 5 to 11, wherein the trigger event includes detecting an alert generated by the medical device.

13. The medical device of any of clauses 5 to 12, further including: a display includes a touch screen; and touch sensors integrated within the touch screen, wherein the trigger event includes detecting a threshold number of touch screen inputs on a location of the touch screen.

14. The medical device of any of clauses 5 to 13, further including: a microphone configured to detect voice input, wherein the trigger event includes detecting one or more keywords from the user.

15. The medical device of clause 14, wherein the trigger event includes detecting a refractory ventricular fibrillation and a keyword within a threshold period of time.

16. The medical device of any of clauses 5 to 15, wherein the input device includes a button, knob, switch, dial, or slider, and the trigger event includes detecting a threshold number of user inputs within a threshold period of time.

17. The medical device of any of clauses 5 to 16, further including: an environmental sensor configured to detect an environmental parameter indicating a state of an environment surrounding the medical device during a rescue event, wherein the trigger event includes a detecting environmental data outside a range.

18. The medical device of clause 17, wherein the environmental sensor includes: a temperature sensor configured to measure a temperature of the environment external to the medical device; or a humidity sensor configured to measure a humidity of the environment external to the medical device.

19. The medical device of any of clauses 5 to 18, further including: a microphone, wherein the processor is further configured to: in response to detecting the trigger event, cause the microphone to capture ambient audio data during a rescue event; cause the memory to store the ambient audio data; and cause the transceiver to transmit the ambient audio data to the external device.

20. The medical device of any of clauses 5 to 19, further including: an accelerometer, wherein the trigger event includes detecting accelerometer data indicating the medical device has been damaged, dropped, or thrown.

21. The medical device of any of clauses 5 to 20, wherein the processor is configured to cause the memory to store the high-fidelity data for a period of time.

22. The medical device of any of clauses 5 to 21, wherein the high-fidelity data includes a plurality of data channels and the processor is further configured to: detect a type of the trigger event, the type including at least one of a medical device error or a user error; and in response to detecting the type of the trigger event, cause the transceiver to transmit a portion of the high-fidelity data to the external device.

23. The medical device of any of clauses 5 to 22, wherein the trigger event includes detecting a first event and a second event within a threshold period of time.

24. The medical device of any of clauses 5 to 23, wherein the external device includes a server associated with a medical device manufacturer.

25. The medical device of any of clauses 5 to 23, wherein the external device includes a computing device within a clinical environment.

26. The medical device of any of clauses 5 to 25, further including: an indicator configured to indicate the medical device is actively recording the high-fidelity data.

27. The medical device of any of clauses 5 to 26, wherein the medical device is a first medical device, the processor is further configured to: determine that the first medical device is communicatively coupled to a second medical device, wherein the trigger event includes determining the first medical device is communicatively coupled to the second medical device.

28. The medical device of any of clauses 5 to 27, wherein the medical device is a first medical device, the processor is further configured to: determine that the first medical device is communicatively coupled to a second medical device, the second medical device includes a microphone; receive audio data from the second medical device; determine that the audio data includes a keyword; and in response to determining the audio data includes the keyword, cause the first medical device to store the audio data in a recorded file.

29. The medical device of any of clauses 5 to 28, wherein the medical device is a first medical device, the processor is further configured to: determine that the first medical device is communicatively coupled to a second medical device; determine the first medical device is associated with a first setting; receive data from the second medical device indicating the second medical device is associated with a second setting; and determine that the first setting and the second setting are incompatible, wherein the trigger event includes determining that the first setting and the second setting are incompatible.

30. The medical device of any of clauses 5 to 29, wherein the trigger event is a first trigger event, the processor further configured to: determine the first trigger event is associated with a first priority level; in response to detecting the first trigger event, cause the memory to store a first portion of the high-fidelity data; detect a second trigger event, the second trigger event associated with a second priority level higher than the first priority level; in response to detecting the second trigger event, cause the memory to store a second portion of the high-fidelity data; and cause the transceiver to transmit the second portion of the high-fidelity data to the external device.

31. The medical device of any of clauses 5 to 30, wherein the processor is further configured to: de-identify the portion of the high-fidelity data; and cause the transceiver to transmit the de-identified data to the external device.

32. The medical device of clause 5, wherein the medical device is a pacemaker and the trigger event includes detecting a threshold number of electrical signals indicating an artifact.

33. The medical device of any of clauses 5 to 32, the processor further configured to: detect an additional trigger event, the additional trigger event causing the medical device to stop storing the high-fidelity data in the memory.

34. The medical device of clause 33, wherein the additional trigger event includes receiving an indication of a selection of a user interface element to stop storing the high-fidelity data.

35. The medical device of clause 33, further including: a monitoring circuit configured to detect an electrocardiogram (ECG) of the subject, wherein the additional trigger event includes receiving data indicating a QRS complex on the ECG.

36. A method, including: continuously recording high-fidelity data at a medical device, the high-fidelity data including at least physiological parameter data, user operational data, and medical device data; detecting a trigger event; in response to detecting the trigger event, storing, in a memory associated with the medical device, a portion of the high-fidelity data corresponding to a period of time after the trigger event; and transmitting the portion of the high-fidelity data to an external device.

37. The method of clause 36, wherein the high-fidelity data includes at least one: data recorded at or above a threshold sample rate; or data recorded at or above a threshold filtering bandwidth.

38. The method of clause 36 or 37, the portion being a first portion, the period of time being a first period of time, wherein the high-fidelity data further includes a second portion corresponding to a second period of time that occurs before detecting the trigger event, wherein the first portion of the high-fidelity data includes display data indicating information visually presented on a display of the medical device and during the trigger event.

39. The method of any of clauses 36 to 38, further including: in response to detecting the trigger event, capturing, via microphone disposed on the medical device, ambient sound data during a rescue event; storing the ambient sound data in the memory associated with the medical device; and transmitting the ambient sound data to the external device.

40. The method of any of clauses 36 to 39, further including: de-identifying the portion of the high-fidelity data; and transmitting the de-identified data to the external device.

41. The method of any of clauses 36 to 40, further including: determining a type of the medical device; and in response to determining the type of the medical device, determining a threshold sample rate of the high-fidelity data.

### Conclusion

The features disclosed in the foregoing description, or the following claims, or the accompanying drawings, expressed in their specific forms or in terms of a means for performing the disclosed function, or a method or process for attaining the disclosed result, as appropriate, may, separately, or in any combination of such features, be used for realizing implementations of the disclosure in diverse forms thereof.

As will be understood by one of ordinary skill in the art, each implementation disclosed herein can comprise, consist essentially of or consist of its particular stated element, step, or component. Thus, the terms “include” or “including” should be interpreted to recite: “comprise, consist of, or consist essentially of.” The transition term “comprise” or “comprises” means has, but is not limited to, and allows for the inclusion of unspecified elements, steps, ingredients, or components, even in major amounts. The transitional phrase “consisting of” excludes any element, step, ingredient or component not specified. The transition phrase “consisting essentially of” limits the scope of the implementation to the specified elements, steps, ingredients or components and to those that do not materially affect the implementation. As used herein, the term “based on” is equivalent to “based at least partly on,” unless otherwise specified.

Unless otherwise indicated, all numbers expressing quantities, properties, conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present disclosure. At the very least, and not as an attempt to limit the application of the doctrine of equivalents to the scope of the claims, each numerical parameter should at least be construed in light of the number of reported significant digits and by applying ordinary rounding techniques. When further clarity is required, the term “about” has the meaning reasonably ascribed to it by a person skilled in the art when used in conjunction with a stated numerical value or range, i.e. denoting somewhat more or somewhat less than the stated value or range, to within a range of ±20% of the stated value; ±19% of the stated value; ±18% of the stated value; ±17% of the stated value; ±16% of the stated value; ±15% of the stated value; ±14% of the stated value; ±13% of the stated value; ±12% of the stated value; ±11% of the stated value; ±10% of the stated value; ±9% of the stated value; ±8% of the stated value; ±7% of the stated value; ±6% of the stated value; ±5% of the stated value; ±4% of the stated value; ±3% of the stated value; ±2% of the stated value; or ±1% of the stated value.

Notwithstanding that the numerical ranges and parameters setting forth the broad scope of the disclosure are approximations, the numerical values set forth in the specific examples are reported as precisely as possible. Any numerical value, however, inherently contains certain errors necessarily resulting from the standard deviation found in their respective testing measurements.

The terms “a,” “an,” “the” and similar referents used in the context of describing implementations (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate implementations of the disclosure and does not pose a limitation on the scope of the disclosure. No language in the specification should be construed as indicating any non-claimed element essential to the practice of implementations of the disclosure.

Groupings of alternative elements or implementations disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Certain implementations are described herein, including the best mode known to the inventors for carrying out implementations of the disclosure. Of course, variations on these described implementations will become apparent to those of ordinary skill in the art upon reading the foregoing description. The inventors expect skilled artisans to employ such variations as appropriate, and the inventors intend for implementations to be practiced otherwise than specifically described herein. Accordingly, the scope of this disclosure includes all modifications and equivalents of the subject matter recited in the claims appended hereto as permitted by applicable law. Moreover, any combination of the above-described elements in all possible variations thereof is encompassed by implementations of the disclosure unless otherwise indicated herein or otherwise clearly contradicted by context.

What is claimed is:

1. A monitor-defibrillator comprising:
a monitoring circuit configured to detect an electrical signal indicative of an electrocardiogram (ECG) of a subject during a rescue event;
a parameter sensor configured to detect a physiological parameter of the subject;
a display configured to output a visual representation of the ECG;
an input device configured to receive an input signal from a user;
a treatment circuit configured to output an electrical shock to a heart of the subject in response to the input device receiving the input signal from the user;
memory comprising a circular buffer with a predetermined storage capacity;
a transceiver; and
a processor configured to:
continuously record, in the circular buffer, high-fidelity data comprising physiological parameter data indicating the physiological parameter, user operational data indicating the input signal, and device data during the rescue event indicating the visual representation of the ECG;
detect a trigger event indicating an anomaly occurred during the rescue event;
in response to detecting the trigger event:
cause the transceiver to transmit, to an external device, a portion of the high-fidelity data recorded for a period of time after the trigger event.

2. The monitor-defibrillator of claim 1, wherein the high-fidelity data comprises:
data recorded at or above a threshold sample rate; or
data recorded at or above a threshold filtering bandwidth.

3. The monitor-defibrillator of claim 1, the portion being a first portion, the period of time being a first period of time, wherein the high-fidelity data further comprises a second portion corresponding to a second period of time that occurs before detecting the trigger event,
wherein the processor is further configured to:
in response to detecting the trigger event:
cause the transceiver to transmit the second portion of the high-fidelity data to the external device.

4. A medical device, comprising:
a physiological sensor configured to detect a physiological parameter of a subject;
an input device configured to detect an input signal from a user;
memory comprising a circular buffer;
a transceiver; and
a processor configured to:
continuously record, in the circular buffer, high-fidelity data comprising physiological parameter data indicative of the physiological parameter, user operational data, or device data;
detect a trigger event; and
in response to detecting the trigger event, cause the transceiver to transmit, to an external device, a portion of the high-fidelity data corresponding to a period of time after the trigger event.

5. The medical device of claim 4, wherein the high-fidelity data comprises the physiological parameter data, and
wherein the physiological parameter comprises an ECG, a capnograph, an end-tidal carbon dioxide (EtCO₂), an airway parameter, a blood oxygenation, a blood flow parameter, a blood pressure, a heart rate, or a temperature.

6. The medical device of claim 4, wherein the high-fidelity data comprises the user operational data, and
wherein the user operational data indicates the input signal from the user.

7. The medical device of claim 4, further comprising:
an output device configured to output a signal to the user,
wherein the high-fidelity data comprises the device data, and
wherein the device data indicates the signal output to the user by the output device.

8. The medical device of claim 4, wherein the processor is configured to detect the trigger event by detecting an artifact in the physiological parameter data.

9. The medical device of claim 4, further comprising:
a display comprising a touch screen; and
touch sensors integrated within the touch screen,
wherein the processor is configured to detect the trigger event by determining that the touch sensors have detected a threshold number of touch screen inputs on a location of the touch screen.

10. The medical device of claim 4, further comprising:
a microphone configured to detect voice input from the user,
wherein the processor is configured to detect the trigger event by detecting a keyword in the voice input.

11. The medical device of claim 4, further comprising:
an environmental sensor configured to detect an environmental parameter indicating a state of an environment surrounding the medical device,
wherein the processor is configured to detect the trigger event by determining that the environmental parameter is outside of a threshold range.

12. The medical device of claim 4, further comprising:
an accelerometer configured to detect an acceleration of the medical device,
wherein the processor is configured to detect the trigger event by detecting, based on the acceleration of the medical device, that the medical device has been damaged, dropped, or thrown.

13. The medical device of claim 4, wherein the processor is further configured to:
in response to detecting the trigger event, cause the memory to store the portion of the high-fidelity data outside of the circular buffer.

14. The medical device of claim 4, wherein the trigger event comprises a medical device error or a user error.

15. The medical device of claim 4, the medical device being a first medical device, wherein the processor is further configured to:
determine that the first medical device is communicatively coupled to a second medical device, and
wherein the trigger event comprises determining the first medical device is communicatively coupled to the second medical device.

16. The medical device of claim 4, the trigger event being a first trigger event, wherein the processor is further configured to:
determine the first trigger event is associated with a first priority level;
in response to detecting the first trigger event, cause the memory to store a first portion of the high-fidelity data outside of the circular buffer;
detect a second trigger event, the second trigger event associated with a second priority level higher than the first priority level;
in response to detecting the second trigger event:
cause the memory to store a second portion of the high-fidelity data outside of the circular buffer; or
cause the transceiver to transmit the second portion of the high-fidelity data to the external device.

17. The medical device of claim 5, wherein the processor is further configured to:
de-identify the portion of the high-fidelity data; and
cause the transceiver to transmit the de-identified data to the external device.

18. A method, comprising:
continuously recording, in a circular buffer, high-fidelity data at a medical device, the high-fidelity data comprising physiological parameter data, user operational data, or medical device data;
detecting a trigger event; and
in response to detecting the trigger event, transmitting, to an external device, a portion of the high-fidelity data corresponding to a period of time before or after the trigger event.

19. The method of claim 18, wherein the high-fidelity data comprises:
data recorded at or above a threshold sample rate; or
data recorded at or above a threshold filtering bandwidth.

20. The method of claim 18, further comprising:
generating de-identified data by de-identifying the portion of the high-fidelity data; and
wherein transmitting the portion of the high-fidelity data comprises transmitting the de-identified data to the external device.
